SEQUENCE LISTING (17 sequences)

SEQ ID NO: 1 - protein, 11 aa, homo sapien
GARRSSWRVVS

SEQ ID NO: 2 - protein, 11 aa, homo sapien
GARRSSWRVIS

SEQ ID NO: 3 - protein, 11 aa, homo sapien
GARRASWRIIS

SEQ ID NO: 4 - protein, 99 aa, homo sapien
MDKNELVQKAKLAEQAERYDDMAACMLSVTEQGAELSNEERNLLSVAYKNVVGARRSSWRVVSSIEQKTEGAEKKQQMAREYREKIETELRDICNDVLS

SEQ ID NO: 5 - protein, 101 aa, homo sapien
MTMDKSELVQKAKLAEQAERYDDMAAAMKAVTEQGHELSNEERNLLSVAYKNVVGARRSSWRVISSIEQKTERNEKKQQMGKEYREKIEAELQDICNDVLE

SEQ ID NO: 6 - protein, 102 aa, homo sapien
MVDREQLVQKARLAEQAERYDDMAAAMKNVTELNEPLSNEERNLLSVAYKNVVGARRSSWRVISSIEQKTSADGNEKKIEMVRAYREKIEKELEAVCQDVLS

SEQ ID NO: 7 - protein, 102 aa, homo sapien
MGDREQLLQRARLAEQAERYDDMASAMKAVTELNEPLSNEDRNLLSVAYKNVVGARRSSWRVISSIEQKTMADGNEKKLEKVKAYREKIEKELETVCNDVLS

SEQ ID NO: 8 - protein, 102 aa, homo sapien
MDDREDLVYQAKLAEQAERYDEMVESMKKVAGMDVELTVEERNLLSVAYKNVIGARRASWRIISSIEQKEENKGGEDKLKMIREYRQMVETELKLICCDILD

SEQ ID NO: 9 - protein, 99 aa, homo sapien
MEKTELIQKAKLAEQAERYDDMATCMKAVTEQGAELSNEERNLLSVAYKNVVGGRRSAWRVISSIEQKTDTSDKKLQLIKDYREKVESELRSICTTVLE

SEQ ID NO: 10 - protein, 101 aa, homo sapien
MERASLIQKAKLAEQAERYEDMAAFMKGAVEKGEELSCEERNLLSVAYKNVVGGQRAAWRVLSSIEQKSNEEGSEEKGPEVREYREKVETELQGVCDTVLG

SEQ ID NO: 11 - protein, 245 aa, homo sapien
MDKNELVQKAKLAEQAERYDDMAACMKSVTEQGAELSNEERNLLSVAYKNVVGARRSSWRVVSSIEQKTEGAEKKQQMAREYREKIETELRDICNDVLSLLEKFLIPNASQAESKVFYLKMKGDYYRYLAEVAAGDDKKGIVDQSQQAYQEAFEISKKEMQPTHPIRLGLALNFSVFYYEILNSPEKACSLAKTAFDEAIAELDTLSEESYKDSTLIMQLLRDNLTLWTSDTQGDEAEAGEGGEN

SEQ ID NO: 12 - protein, 247 aa, homo sapien
MVDREQLVQKARLAEQAERYDDMAAAMKNVTELNEPLSNEERNLLSVAYKNVVGARRSSWRVISSIEQKTSADGNEKKIEMVRAYREKIEKELEAVCQDVLSLLDNYLIKNCSETQYESKVFYLKMKGDYYRYLAEVATGEKRATVVESSEKAYSEAHEISKEHMQPTHPIRLGLALNYSVFYYEIQNAPEQACHLAKTAFDDAIAELDTLNEDSYKDSTLIMQLLRDNLTLWTSDQQDDDGGEGNN

SEQ ID NO: 13 - protein, 246 aa, homo sapien
MTMDKSELVQKAKLAEQAERYDDMAAAMKAVTEQGHELSNEERNLLSVAYKNVVGARRSSWRVISSIEQKTERNEKKQQMGKEYREKIEAELQDICNDVLELLDKYLIPNATQPESKVFYLKMKGDYFRYLSEVASGDNKQTTVSNSQQAYQEAFEISKKEMQPTHPIRLGLALNFSVFYYEILNSPEKACSLAKTAFDEAIAELDTLNEESYKDSTLIMQLLRDNLTLWTSENQGDEGDAGEGEN

SEQ ID NO: 14 - protein, 246 aa, homo sapien
MGDREQLLQRARLAEQAERYDDMASAMKAVTELNEPLSNEDRNLLSVAYKNVVGARRSSWRVISSIEQKTMADGNEKKLEKVKAYREKIEKELETVCNDVLSLLDKFLIKNCNDFQYESKVFYLKMKGDYYRYLAEVASGEKKNSVVEASEAAYKEAFEISKEQMQPTHPIRLGLALNFSVFYYEIQNAPEQACLLAKQAFDDAIAELDTLNEDSYKDSTLIMQLLRDNLTLWTSDQQDEEAGEGN

SEQ ID NO: 15 - protein, 255 aa, homo sapien
MDDREDLVYQAKLAEQAERYDEMVESMKKVAGMDVELTVEERNLLSVAYKNVIGARRASWRIISSIEQKEENKGGEDKLKMIREYRQMVETELKLICCDILDVLDKHLIPAANTGESKVFYYKMKGDYHRYLAEFATGNDRKEAAENSLVAYKAASDIAMTELPPTHPIRLGLALNFSVFYYEILNSPDRACRLAKAAFDDAIAELDTLSEESYKDSTLIMQLLRDNLTLWTSDMQGDGEEQNKEALQDVEDENQ

SEQ ID NO: 16 - protein, 245 aa, homo sapien
MEKTELIQKAKLAEQAERYDDMATCMKAVTEQGAELSNEERNLLSVAYKNVVGGRRSAWRVISSIEQKTDTSDKKLQLIKDYREKVESELRSICTTVLELLDKYLIANATNPESKVFYLKMKGDYFRYLAEVACGDDRKQTIDNSQGAYQEAFDISKKEMQPTHPIRLGLALNFSVFYYEILNNPELACTLAKTAFDEAIAELDTLNEDSYKDSTLIMQLLRDNLTLWTSDSAGEECDAAEGAEN

SEQ ID NO: 17 - protein, 248 aa, homo sapien [sequence omitted]

(19) United States
(12) Patent Application Publication
(10) Pub. No.: US 2017/0173053 A1
(43) Pub. Date: Jun. 22, 2017

(54) Method of Modulating Protein 14-3-3 Functionality By Facilitating or Inhibiting Phosphorylation

(71) Applicant: CENTRAL ADELAIDE LOCAL HEALTH NETWORK INC., Adelaide (AU)

(72) Inventors: Joanna Woodcock, Crafers West (AU); Stuart Pitson, Glenelg (AU); Angel Lopez, Medindie (AU)

(21) Appl. No.: 15/237,548

(22) Filed: Aug. 15, 2016

Related U.S. Application Data
(63) Continuation of application No. 13/638,562, filed on May 10, 2013, now abandoned.

(30) Foreign Application Priority Data
Mar. 29, 2010 (AU) 2010901329

(51) Int. Cl. A61K 31/661; A61K 31/133; C12Q 1/48; A61K 31/16; A61K 31/201; A61K 31/132; A61K 31/14

(52) U.S. Cl. CPC A61K 31/661; A61K 31/132; A61K 31/133; A61K 31/14; A61K 31/16; A61K 31/201; C12Q 1/485; G01N 2500/02; G01N 2500/20

(57) ABSTRACT

The present invention relates to a method of modulating cellular activity. More particularly, the present invention provides a method of modulating apoptosis by modulating protein 14-3-3 phosphorylation and, thereby, its functionality. The present invention still further extends to methods for identifying agents capable of modulating protein 14-3-3 phosphorylation. The method and molecules of the present invention are useful, inter alia, in the treatment and/or prophylaxis of conditions characterised by unwanted cellular activity, such as unwanted cell survival.

### FIELD OF THE INVENTION

The present invention relates to a method of modulating cellular activity. More particularly, the present invention provides a method of modulating apoptosis by modulating protein 14-3-3 phosphorylation and, thereby, its functionality. The present invention still further extends to methods for identifying agents capable of modulating protein 14-3-3 phosphorylation. The method and molecules of the present invention are useful, inter alia, in the treatment and/or prophylaxis of conditions characterised by unwanted cellular activity, such as unwanted cell survival.

### BACKGROUND OF THE INVENTION

Bibliographic details of the publications referred to by author in this specification are collected alphabetically at the end of the description.

The reference in this specification to any prior publication (or information derived from it), or to any matter which is known, is not, and should not be taken as an acknowledgment or admission or any form of suggestion that that prior publication (or information derived from it) or known matter forms part of the common general knowledge in the field of endeavour to which this specification relates.

As used herein, the term “derived from” shall be taken to indicate that a particular integer or group of integers has originated from the species specified, but has not necessarily been obtained directly from the specified source. Further, as used herein the singular forms of “a”, “and” and “the” include plural referents unless the context clearly dictates otherwise.

The 14-3-3 proteins are a conserved family of dimeric phospho-serine binding proteins that interact and modulate the functions of multiple cellular proteins and in so doing regulate many signalling pathways (Tzivion and Avruch 2002, J. Biol. Chem. 277:3061-64; Fu et al. 2000, Ann. Rev. Pharma. Tox. 40:617-47). To date, one of the most clearly defined roles for 14-3-3 proteins is protecting cells from apoptosis (Masters and Fu 2001, J. Biol. Chem. 276:45193-45200; Xing et al. 2000, EMBO J. 19:349-58). In particular, the binding of 14-3-3 to pro-apoptotic proteins curbs their apoptotic behaviour: the interaction of 14-3-3 with BAD retains BAD in the cytosol, thereby preventing its interaction with the mitochondrial protein, Bel-X_{L} and induction of apoptosis (Zha et al. 1996, Cell 87:619-628) and through binding to ASK-1 14-3-3 negatively regulates its kinase activity and blocks downstream SAPK activation (Zhang et al. 1999, PNAS USA 96:8511-15). The 14-3-3 proteins are composed of two 30kDa monomer units that are each capable of binding a phospho-serine motif via an amphipathic groove. Dimers of 14-3-3 are formed by the N-terminal α helices, with helix 1 of one monomer interacting with helices 3 and 4 of another. Functionally, 14-3-3 proteins perform multiple roles in regulating cellular protein activities and importantly, these functions of 14-3-3 are dependent on its dimeric structure. (Xing et al. 2000, supra; Yaffe 2002, FEBS Letts. 513:53-57). For example, a non-dimerising mutant of 14-3-3 does not support efficient Raf-1 activation (Tzivion et al. 1998, Nature 394:88-92).

Sphingosine is a basic lipid, generated from the breakdown of plasma membrane sphingomyelin and ceramide (Cuvillier 2002, supra). Accumulation of sphingosine in cells is closely associated with the induction of apoptosis by physiological activators (Cuvillier 2002, supra) and indeed this lipid induces apoptosis in many cell types in its own right (Igarashi 1997, J. Biochem. 122:1080-87) but to date the downstream mechanism involved in sphingosine-induced signalling has remained elusive. Sphingosine has been shown to modulate the activities of several kinases (Smith et al. 2000, Methods Enz. 312:361-373; Davis et al. 1988, J. Biol. Chem. 263:5373-79; McDonald et al. 1991, J. Biol. Chem. 266:21773-776; Bokoch et al. 1998, J. Biol. Chem. 273:8137-44; King et al. 2000, J. Biol. Chem. 275:18108-18113) and these effects may contribute to the apoptotic activity of sphingosine. In this regard it has previously been shown that sphingosine activates protein kinase A (PKA) type II both in vitro and in vivo.

However, to date it had been understood that means for achieving phosphorylation of 14-3-3 depended on the activation of PKA and that contacting 14-3-3 with activated PKA would effect 14-3-3 phosphorylation. However, it has been determined that, in fact, not all forms of activated PKA can effect 14-3-3 phosphorylation. In work leading up to the present invention it has been determined that it is binding of sphingosine to 14-3-3 which is central to thereafter effecting 14-3-3 phosphorylation. Further, the features and characteristics of sphingosine or a derivative, equivalent or mimetic thereof which effect binding to and enable phosphorylation of 14-3-3 have been determined. An assay for screening for or testing the functionality of a molecule in this regard has also been developed. These findings have therefore enabled the development of methods for regulating 14-3-3 phosphorylation and thereby regulating cellular functionality, in particular cellular apoptosis.

### SUMMARY OF THE INVENTION

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps.

The subject specification contains amino acid sequence information prepared using the programme Patentin Version 3.1, presented herein after the bibliography. Each amino acid sequence is identified in the sequence listing by the numeric indicator <201> followed by the sequence identifier (eg. <210>1, <210>2, etc). The length, type of sequence (protein, etc) and source organism for each amino, acid sequence is indicated by information provided in the numeric indicator fields <211>, <212> and <213>, respectively. Nucleotide sequences referred to in the specification are identified by the indicator SEQ ID NO: followed by the sequence identifier (eg. SEQ ID NO:1, SEQ ID NO:2, etc.). The sequence identifier referred to in the specification correlates to the information provided in numeric indicator field <400> in the sequence listing, which is followed by the sequence identifier (eg. <400>1, <400>2, etc). That is SEQ ID NO:1 as detailed in the specification correlates to the sequence indicated as <400>1 in the sequence listing. In terms of the 14-3-3 protein, the amino acid residue serine 58 is numbered in the context of the 14-3-3ζ motif GARRSSWRVVS (SEQ ID NO:1), which itself corresponds to residues 53-63. Accordingly the S residue which is shown in bold corresponds to Ser58. 14-3-3 β/η/γ exhibit the equivalent residue in the context of the motif GARRSSWRVIS (SEQ ID NO:2) while 14-3-3ε exhibits the equivalent serine residue in the context of the motif GARRASWRIIS (SEQ ID NO:3). With regard to the identification of the protein 14-3-3 sphingosine binding cleft, the residues R18, D20, D21, R55, K85 and E89 are depicted in FIG. 12. This figure provides an alignment of the relevant section of the seven protein 14-3-3 isoform sequences. The numbering of these 6 residues corresponds to the numbering of the protein 14-3-3ζ isoform. The equivalent sequence regions of 6 other protein 14-3-3 isoforms are aligned with the protein 14-3-3ζ isoform in FIG. 12 such that the position of the relevant corresponding residues are also aligned. FIG. 7 provides the full sequence for each of the protein 14-3-3 isoforms and indicates the actual numbering of the residues which correspond to residues R18, D20, D21, R55, K85 and E89 of protein 14-3-3ζ.

One aspect of the present invention provides a method of modulating protein 14-3-3 or homologue or variant functionality, said method comprising contacting said protein 14-3-3 with an agent which either modulates the interaction of sphingosine or homologue or variant with said protein 14-3-3 or which mimics the sphingosine interaction wherein agonising said interaction facilitates phosphorylation of Ser58 or analogous residue thereby inhibiting the functionality of said protein 14-3-3 and wherein antagonising said interaction inhibits phosphorylation of Ser58 or analogous residue thereby maintaining said protein 14-3-3 functionality.

In another aspect there is provided a method of modulating protein 14-3-3 or homologue or variant functionality in a mammal said method comprising administering to said mammal an effective amount of an agent for a time and under conditions sufficient to either modulate the interaction of sphingosine or homologue or variant with protein 14-3-3 or to mimic the sphingosine interaction wherein agonising said interaction facilitates phosphorylation of Ser58 or analogous residue thereby inhibiting the functionality of said protein 14-3-3 and wherein antagonising said interaction inhibits phosphorylation of Ser58 or analogous residue thereby maintaining said protein 14-3-3 functionality.

In still another aspect there is provided a method of modulating cellular functioning in a mammal said method comprising administering to said mammal an effective amount of an agent for a time and under conditions sufficient to modulate the interaction of sphingosine or homologue or variant with protein 14-3-3 or to mimic the sphingosine interaction wherein agonising said interaction facilitates phosphorylation of Ser58 or analogous residue thereby inhibiting the functionality of said protein 14-3-3 and wherein antagonising said interaction inhibits phosphorylation of Ser58 or analogous residue thereby maintaining said protein 14-3-3 functionality.

In yet another aspect said serine 58 or analogous residue is selected from the list consisting essentially of:
(i) Ser58 of protein 14-3-3λ;
(ii) Ser59 of protein 14-3-3η;
(iii) Ser59 of protein 14-3-3γ;
(iv) Ser59 of protein 14-3-3ε; and
(v) Ser60 of protein 14-3-3β.

In still another aspect the cellular functioning which is the subject of modulation is cell division, cell growth or cellular apoptosis.

In a related aspect there is provided a method for detecting an agent which facilitates the phosphorylation of protein 14-3-3 at Ser58 or analogous residue said method comprising contacting a putative agent with protein 14-3-3 and a serine kinase or catalytic subunit thereof and screening for protein 14-3-3 phosphorylation at Ser58 or analogous residue.

In another related aspect there is provided a method for detecting an agent which antagonises the phosphorylation of protein 14-3-3 at Ser58 or analogous residue said method comprising:
(i) contacting a putative agent with protein 14-3-3 and sphingosine or functional equivalent;
(ii) contacting the composition of (i) with a serine kinase or catalytic subunit thereof; and screening for protein 14-3-3 phosphorylation at Ser58 or analogous residue wherein a reduction in the level of phosphorylation relative to a control level is indicative of the antagonistic activity of said putative agent.

In a further aspect there is provided a method for detecting an agent which facilitates the phosphorylation of protein 14-3-3 at Ser 58 or analogous residue said method comprising contacting a putative agent with protein 14-3-3 and protein kinase A or catalytic subunit thereof and screening for protein 14-3-3 phosphorylation at Ser58 or analogous residue.

In another aspect there is provided a method for detecting an agent which antagonises the phosphorylation of protein 14-3-3 at Ser58 or analogous residue said method comprising:
(i) contacting a putative agent with protein 14-3-3 and sphingosine or functional equivalent;
(ii) contacting the composition of (i) with protein kinase A or catalytic subunit thereof; and screening one or both of for protein 14-3-3 phosphorylation at Ser58 or analogous residue or protein 14-3-3 ubiquitination wherein a reduction in the level of phosphorylation relative to a control level is indicative of the antagonistic activity of said putative agent.

In yet another aspect said agent which agonises the interaction of sphingosine with protein 14-3-3 is a sphingosine mimetic, which agent interacts with one or more of protein 14-3-3 residues D20, D21, E89, K85, R18, E5, K74 or R55 or analogous residue. More specific examples of such agents are provided hereafter in the context of the class of agents represented by formula (I).

In a further aspect said protein 14-3-3 is 14-3-3ζ and said residues are D20, D21, E89, K85, R18, E5, K74 or R55.

In another aspect, said protein 14-3-3 is selected from:

(i) protein 14-3-3γ and said residues are R19, D21, D22, R56, K88 or E92;

(ii) protein 14-3-3β and said residues are R20, D22, D23, R57, K87 or E91;

(iii) protein 14-3-3η and said residues are R19, D21, D22, R56, K88 or E92;

(iv) protein 14-3-3ε and said residues are R19, D2I, E22, R56 or E92;

(v) protein 14-3-3τ and said residues are R18, D20, D21, R55, K85 or E89;

(vi) protein 14-3-3σ and said residues are R18, E20, D21, K87 or E91.

In yet another aspect, said residues are D20, D21, E89, K85 or R18.

In still another aspect, said agent is non-phosphorylated.

In yet still another aspect of the present invention, the assay hereinbefore described has been performed to identify agonist molecules and sphingosine mimetics. To this end, reference to “agonist” should be understood to include reference to sphingosine mimetics. In accordance with these findings, said agent may be represented by formula (I):

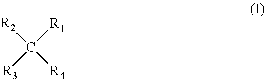

R₁ represents a saturated or unsaturated, branched or linear optionally substituted C₆-C₂₈aliphatic group;
R₂ represents a C₂-C₄ alkyl substituted from 1 to 3 times with groups independently selected from
   —(CH₂)ₘOR′ where R′ is H or C₁₋₅ alkyl, —(CH₂)ₘCOOR″ where R″ is H or C₁₋₅ alkyl, —(CH₂)ₘNH₂, —(CH₂)ₘNH₃, —(CH₂)ₘN(C₁₋₆alkyl)₂, —(CH₂)ₘCHNH(COR′″)—(CH₂)ₚOH, —(CH₂)ₘN(C₁₋₆alkyl)₃, —(CH₂)ₘP(O)(OH)₂, —(CH)(—NHC(O)—C₁-C₂₈alkyl)-(CH₂)ₚ—O—P(O)(OH)—O—(CH₂)ₘ—NH₃, and —(CH)(—NHC(O)—C₁-C₂₈alkyl)-(CH₂)ₚ—O—P(O)(OH)—O—(CH₂)ₘ—N(C₁-C₄alkyl)₃;
R₃ and R₄ independently represent H or —(CH₂)ₙOR′″ where R′″ is H or C₁₋₃ alkyl;

m is 0, 1, or 2;

n is 0, 1, or 2;

p is 1 or 2;

or a pharmaceutically acceptable salt thereof.

Specific examples of possible agents of the present invention include:

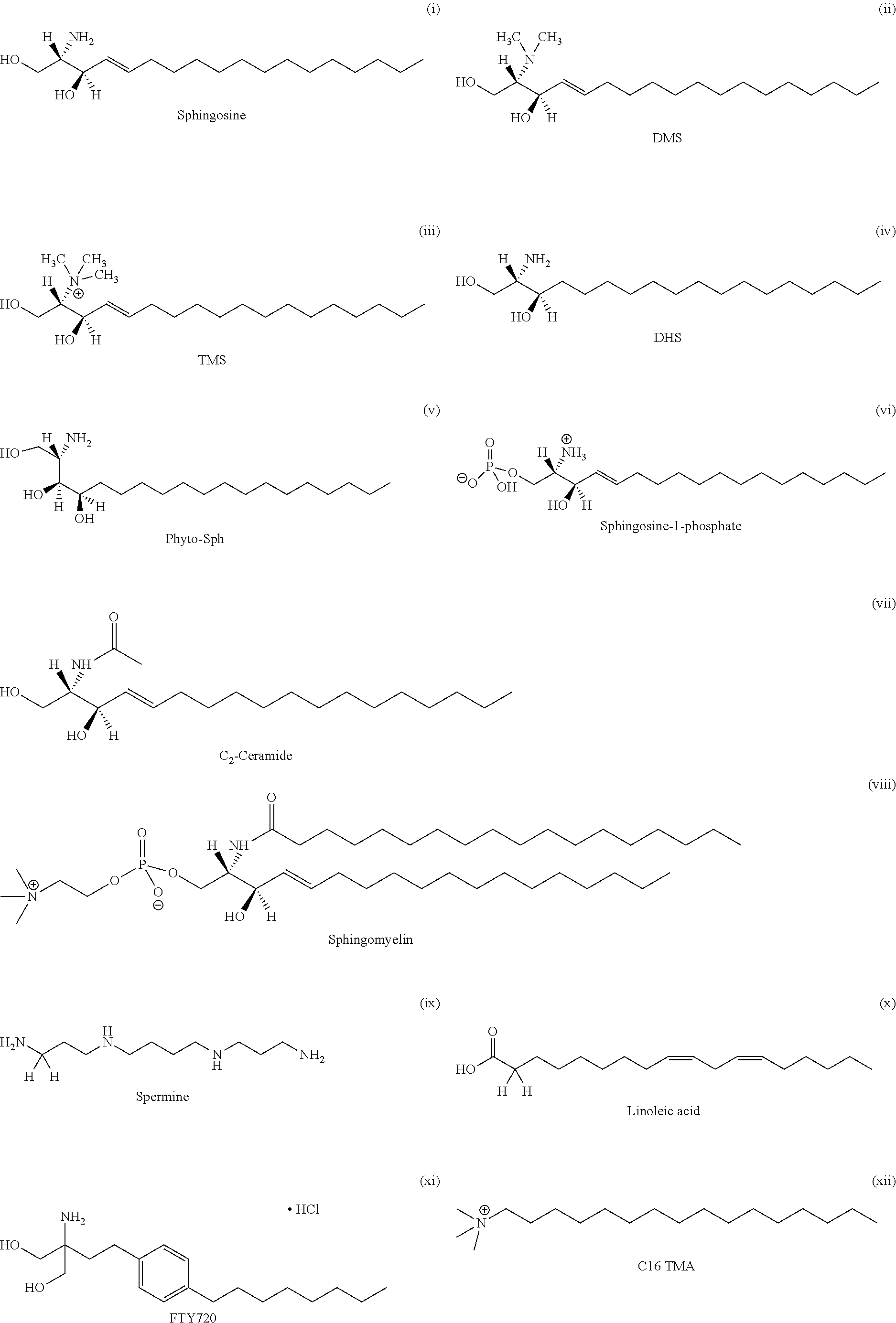

Yet another aspect of the present invention is directed to a method for the treatment or prophylaxis of a condition in a mammal, which condition is characterised by inappropriate protein 14-3-3 or homologue or variant functionality said method comprising administering to said mammal an effective amount of an agent which either modulates the interaction of sphingosine or homologue or variant with said protein 14-3-3 or which mimics the sphingosine interaction wherein agonising said interaction facilitates phosphorylation of Ser58 or analogous residue thereby inhibiting the functionality of said protein 14-3-3 and wherein antagonising said interaction inhibits phosphorylation of Ser58 or analogous residue thereby maintaining said protein 14-3-3 functionality.

In yet another aspect, the present invention is directed to a method for regulating cell division, cell growth or cellular apoptosis in a mammal said method comprising administering to said mammal an effective amount of an agent which either modulates the interaction of sphingosine or homologue or variant with protein 14-3-3 or which mimics the sphingosine interaction wherein agonising said interaction facilitates phosphorylation of Ser58 or analogous residue thereby inhibiting the functionality of said protein 14-3-3 and wherein antagonising said interaction inhibits phosphorylation of Ser58 or analogous residue thereby maintaining said protein 14-3-3 functionality.

In still another aspect there is provided a method for treating a condition characterised by unwanted cellular division, said method comprising administering to said mammal an effective amount of an agent which either agonises the interaction of sphingosine or homologue or variant with protein 14-3-3 or homologue or variant or mimics the sphingosine interaction wherein agonising said interaction facilitates phosphorylation of Ser58 or analogous residue thereby inducing cellular apoptosis.

In one aspect, said condition is a neoplastic condition.

In another aspect there is provided a method for treating a condition characterised by unwanted cell death, said method comprising administering to said mammal an agent which antagonises the interaction of sphingosine or homologue or variant with protein 14-3-3 or homologue or variant wherein antagonising said interaction inhibits phosphorylation of Ser58 or analogous residue thereby inhibiting cellular apoptosis.

In still another aspect, the present invention is directed to an agent for use in modulating protein 14-3-3 or homologue or variant functioning wherein agonising or mimicking the interaction of sphingosine with protein 14-3-3 facilitates phosphorylation of Ser58 or analogous residue thereby inhibiting the functionality of said protein 14-3-3 and wherein antagonising said interaction inhibits phosphorylation of Ser58 or analogous residue thereby maintaining said protein 14-3-3 functionality.

In yet another aspect, the present invention is directed to an agent for use in regulating cell division, cell growth, cell death or cellular apoptosis in a mammal, which agent either modulates the interaction of sphingosine or homologue or variant with protein 14-3-3 or mimics the sphingosine interaction wherein agonising said interaction facilitates phosphorylation of Ser58 or analogous residue thereby inhibiting the functionality of said protein 14-3-3 and wherein antagonising,said interaction inhibits phosphorylation of Ser58 or analogous residue thereby maintaining said protein 14-3-3 functionality.

In still another aspect there is provided the use of an agent in the manufacture of a medicament for treating a condition characterised by unwanted cellular division, which agent agonises the interaction of sphingosine or homologue or variant with protein 14-3-3 or homologue or variant or mimics the sphingosine interaction wherein agonising said interaction facilitates phosphorylation of Ser58 or analogous residue thereby inducing apoptosis.

In yet still another aspect there is provided the use of an agent in the manufacture of a medicament for treating a condition characterised by unwanted cell death, which agent antagonises the interaction of sphingosine with protein 14-3-3 or homologue or variant wherein antagonising said interaction inhibits phosphorylation of Ser58 or analogous residue thereby inhibiting apoptosis.

In yet another further aspect, the present invention contemplates a pharmaceutical composition comprising the modulatory agent as hereinbefore defined together with one or more pharmaceutically acceptable carriers and/or diluents. These agents are referred to as the active ingredients.

Yet another aspect of the present invention relates to the agent as hereinbefore defined or as identified in accordance with the screening method hereinbefore described, when used in the method of the present invention.

Single and three letter abbreviations used throughout the specification are defined in Table 1.

TABLE 1
Single and three letter amino acid abbreviations

| | Three-letter | One-letter |
|---|---|---|
| Amino Acid | Abbreviation | Symbol |
| Alanine | Ala | A |
| Arginine | Arg | R |
| Asparagine | Asn | N |
| Aspartic acid | Asp | D |
| Cysteine | Cys | C |
| Glutamine | Gln | Q |
| Glutamic acid | Glu | E |
| Glycine | Gly | G |
| Histidine | His | H |
| Isoleucine | Ile | I |
| Leucine | Leu | L |
| Lysine | Lys | K |
| Methionine | Met | M |
| Phenylalanine | Phe | F |
| Proline | Pro | P |
| Serine | Ser | S |
| Threonine | The | T |
| Tryptophan | Trp | W |
| Tyrosine | Tyr | Y |
| Valine | Val | V |
| Any residue | Xaa | X |

### BRIEF DESCRIPTION OF THE DRAWINGS

FIG. 1 is an image depicting that sphingosine is required for 14-3ζ phosphorylation on Ser58 by the catalytic subunit of PKA. 14-3-3ζ (wild-type or S58A mutant) was incubated with the catalytic subunit of PKA in the presence (+) or absence (−) of 50 μM sphingosine (A) or DMS (B) delivered in 0.1% ethanol. Phosphorylated l4-3ζ was detected by autoradiography. Gels were coomassie stained to show equal loading of 14-3-3 substrate. The figure shows a representative result of multiple experiments.

FIG. 2 is an image depicting that sphingosine modulates (A) Dose-response of 14-3-3 phosphorylation induced by DMS. The assay was carried out by incubating 14-3-3ζ (0.5 μg) with catalytic subunit of PKA (0.2 Units) with the indicated concentration of DMS delivered in 0.1% ethanol. Reactions were separated on SDS-PAGE and phosphorylated protein detected by autoradiography. Coomassie staining of the gel indicates equal loading of 14-3-3ζ. (B) 14-3-3ζ was incubated with the catalytic subunit of PKA and 50 μM D-erythro-sphingosine (Sph), D-erythro-trimethyl-sphingosine (TMS), D-erythro-dihydro-sphingosine (DHS), phyto-sphingosine (PSph), D-erythro-sphingosine-1-phosphate (S1P), C₂-ceramide (C₂-Cer), sphingomyelin (SM), spermine (Sper) or linoleic acid (LA), all delivered in 0.1% ethanol. Phosphorylated 14-3-3ζ was detected by autoradiography. Coomassie staining of the gel indicates equal loading of 14-3-3ζ.

FIG. 3 is an image depicting that sphingosine is required for the phosphorylation of multiple 14-3-3 isoforms in the third helix. (A) Primary sequence alignment of 14-3-3 isoforms in the third helix showing conservation of a Ser phosphorylation site (shown in bold). Asterisks indicate identical amino acids. (B) Recombinant 14-3-3 isoforms were incubated with PKA catalytic subunit in the presence of 50 μM sphingosine, dimethyl-sphingosine (DMS) or FTY 720 (FTY) delivered in 0.1% ethanol (EtOH). Phosphorylated 14-3-3s were detected by autoradiography. Gels were coomassie stained to show equal loading of 14-3-3 substrate.

FIG. 4 is a graphical representation depicting that sphingosine binds to 14-3-3 in vitro. 10 μg of recombinant 14-3-3 was incubated for 15 minutes at room temperature in 50 μl of buffer either in the absence (Fig. A, thick line) or presence of [³H]-sphingosine (0.5 μCi) with 25 μM DMS (thin line). After incubation, the mixtures were separated by gel filtration as described under “examples”. (A) Absorbance at 280 nm was monitored continuously to identify the elution position of 14-3-3 protein. An aliquot of each eluted fraction was analysed for (B) sphingosine-binding to 14-3-3 by measuring [³H]-sphingosine radioactivity as determined by scintillation counting; and (C) 14-3-3 phosphorylatability by incubation with 0.2 Units of PKA catalytic subunit and [³²P]γ-ATP. Phosphorylated 14-3-3 was detected by autoradiography. The chromatography and assays were repeated twice and similar results obtained.

FIG. 5 is an image depicting that DMS is required for phosphorylation of 14-3-3ζ by PKCδCat but not for activation of the kinase. COS cells were transfected with either an empty expression construct (Mock) or one encoding a HA-tagged truncated form of PKCδ corresponding to the caspase-3 cleaved protein (PKCδCat-HA). 20 hours after transfection the cells were lysed and PKCδCat-HA was immunoprecipitated using an anti-HA antibody. (A) PKC assays were carried out on the immunoprecipitates from mock (open bars) and PKCδCat-HA (shaded bars) expressing cells using Ser25 peptide as substrate with [³²P]γ-ATP in the presence or absence of 100 μM DMS (delivered in 0.1% ethanol) or 10 μM of GF109203X (GFX), the PKC inhibitor. The values represent means±S.E. of triplicate determinations. (B) PKCδCat-HA immunoprecipitates were incubated with recombinant 14-3-3ζ and [³²P]γ-ATP in the presence or absence of DMS or GF109203X (GFX). Phosphorylated 14-3-3 was detected by autoradiography after separation of SDS-PAGE (upper panel). The gel was also coomassie stained to show equal loading of 14-3-3 protein (lower panel).

FIG. 6 is an image depicting that sphingosine kinase controls the phosphorylation of 14-3-3 by PKA. (A) COS cells were transfected with an expression construct encoding 14-3-3ζ-Myc and 30 hours after transfection the cells were lysed and 14-3-3 immunoprecipitated using anti-Myc antibody as described under “examples”. Immunoprecipitates were then treated either with or without sphingosine kinase 1 (SKI) prior to analysis of 14-3-3 phosphorylatability by incubating with [³²P]γ-ATP and PKA catalytic subunit (PKA) in the presence or absence of 50 μM DMS as indicated. Phosphorylated 14-3-3 was detected by autoradiography after separation by SDS-PAGE (upper panel). The gel was also coomassie stained to show equal loading of substrate 14-3-3 protein (lower panel). The experiment was repeated with similar results. (B) HEK293 stably transfected with an inducible expression construct for SKI were cultured for 16 hours with (Induced) and without (Un-induced) 100 ng/ml doxycycline. Lipid and cell extracts were prepared as described under “examples”. The sphingosine levels were determined (upper panel) and the induction of SK1 confirmed by immunoblotting (upper panel, inset; U, un-induced and I, induced). The 14-3-3 phosphorylatability was analysed by incubation with PKA catalytic subunit, separation by SDS-PAGE and subsequent immunoblotting with a phospho-specific Ser58 antibody (middle panel). Equal loading of 14-3-3 protein was confirmed by immunoblotting with anti-14-3-3 antibody (lower panel).

FIG. 7 is an image depicting that sphingosine binds to 14-3-3 under conditions of physiological sphingosine accumulation. Jurkat cells stably expressing either 14-3-3ζ-Myc-IRES-GFP or IRES-GFP alone (Vector) were serum-starved and stimulated with anti-CD95 (50 ng/ml) to induce a Fas response as described previously (Cuvillier et al. 2000, J. Biol. Chem. 275:15691-700). Cells were harvested at time points after stimulation (as indicated) and lysates prepared in duplicate. Immunoprecipitation was carried out using μMacs anti-Myc microbeads and the eluted material subjected to sphingosine determination (upper panel) and immunoblotting with anti-Myc (lower panel) to detect 14-3-3ζ Myc. Minimal amounts of sphingosine were associated with material immunoprecipitated from cells transfected with the IRES-GFP construct (open bars) compared with 14-3-3ζ-Myc-IRES-GFP expressing cells (shaded bars).

FIG. 8 demonstrates that Jurkat cells expressing non-phosphorylatable 14-3-3 exhibit slower commitment to apoptosis in response to DMS and FTY720. (A) Immunoblotting of lysates from either parental Jurkat cells (Jurkat) or cells transduced with lentivirus encoding either wild type (Wt) or S58A 14-3-3ζ-Myc with either an anti-14-3-3ζ specific antibody (upper panel) or an anti-pan 14-3-3 antibody (middle panel) to compare the expression level of transduced protein. Myc immunoprecipitates were also immunoblotted with an anti-phospho 14-3-3 binding site antibody (lower panel) to detect proteins associated with 14-3-3ζ-Myc. Endogenous 14-3-3 is indicated by an open arrow and exogenous 14-3-3ζ-Myc with a closed arrow. (B) Lysates from either wild type (Wt) or S58A 14-3-3ζ-Myc expressing Jurkat cells were subjected to in vitro PKA phosphorylation in the presence (+) or absence (−) of 50 μM DMS and the samples immunoblotted after SDS-PAGE separation With either anti-Myc antibody (upper panel), anti-14-3-3ζ antibody (middle panel) or anti-phospho Ser58 14-3-3ζ antibody (lower panel). (C) Serum-starved Jurkat cells were treated with 2 μM FTY720 for 4 h prior to staining with TMRE and NucView™. Forward-versus side-scatter plots (FS versus SS) show the viable cell populations for FTY720 and EtOH (vehicle) treated cells which are analysed in the lower histograms for caspase activity (NucView™) versus ΔΨ_{M} (TMRE). (D and E) Jurkat cell lines expressing either wild type or S58A I4-3-3ζ-Myc by lentiviral transduction were serum-starved for 2 h and treated with either 0.1% ethanol vehicle alone (EtOH), 2 μM dimethyl-sphingosine (DMS), or 2 μM FTY720. Cells were analysed by TMRE staining at 4 h (D) or at the time points indicated (E). (D) Representative analyses of TMRE staining with forward-versus side-scatter plots inset to show the similarity in cell populations. (E) Graphical representation of TMRE analysis over time with open symbols representing vehicle (0.1% EtOH) treated cells and closed symbols representing FTY720 treated cells. Square symbols represent Jurkat cells expressing wild type and triangles represent S58A 14-3-3ζ-Myc expressing cells. The data shown is representative of several experiments.

FIG. 9 A depicts a side view of 14-3-3 in space filling representation indicating the orientation of view B. The monomer units (in green and orange) and interacting phospho—peptides, (in cyan) bound in the dual amphipathic grooves are shown. B. Bottom-up view of 14-3-3 with the location of S58 phosphorylation site (indicated by arrows and asterisk) and key surface acidic (in yellow) and basic (in blue) residues shown. C. In vitro phosphorylation of 14-3-3ζ wild type and mutants as indicated by catalytic PKA subunit in the presence of increasing concentrations of FTY720. Phosphorylation is detected after SDS-PAGE by [³²P]-yATP incorporation and Coomassie staining of gels shows equal 14-3-3 protein loading. D. Mutant 14-3-3 (as indicated) was analysed by native-PAGE and Coomassie staining. The arrow indicates the migration of dimeric 14-3-3 protein. E. A schematic representation of a 14-3-3 dimer showing the buried S58 phosphorylation site in pink, D20, the residue proposed to interact with sphingolipid in yellow and the predicted salt bridge interactions involved in dimerisation. F. & G. In silico docking of FTY720 (shown in cyan in F.) and sphingosine (in pink in G.) in 14-3-3ζ dimer interface.

FIG. 10 is an image depicting that C₁₆ and C₁₄ trimethyl-ammonium (TMA) compounds modulate 14-3-3 for phosphorylation. (A) Chemical structures and names of TMA molecules. (B) Recombinant 14-3-3 (0.5 μg) was incubated with PKA catalytic subunit in the presence of either 125 μM or 62.5 μM TMA as indicated delivered in 0.1% ethanol. Phosphorylated 14-3-3 was detected either by spotting the reaction onto P81 paper and scintillation counting after extensive washing with 0.75% orthophosphoric acid (shown graphically) or by autoradiography (lower panel).

FIG. 11. Long trimethyl-ammonium (TMA) molecules target 14-3-3 for phosphorylation and induce mitochondrial-mediated apoptosis. A. Chemical structures of TMAs. B. Effect of TMAs on PKA-mediated 14-3-3 phosphorylation. C. Effect of TMAs on Jurkat cells after 16 hours incubation assessed by ΔΨM (TMRE) and caspase 3 activity (NucView™) analysis. D. Jurkat cells over-expressing Bc1-2 are protected from apoptosis induced by CTAB. Forward-side-scatter histograms showing cell viability are inset and caspase 3 activity as assessed by NucView™ analysis of viable cells shown. E. Examples of TMA-derivatives of FTY720 that will be analysed for effects on 14-3-3 dimer stability and apoptosis.

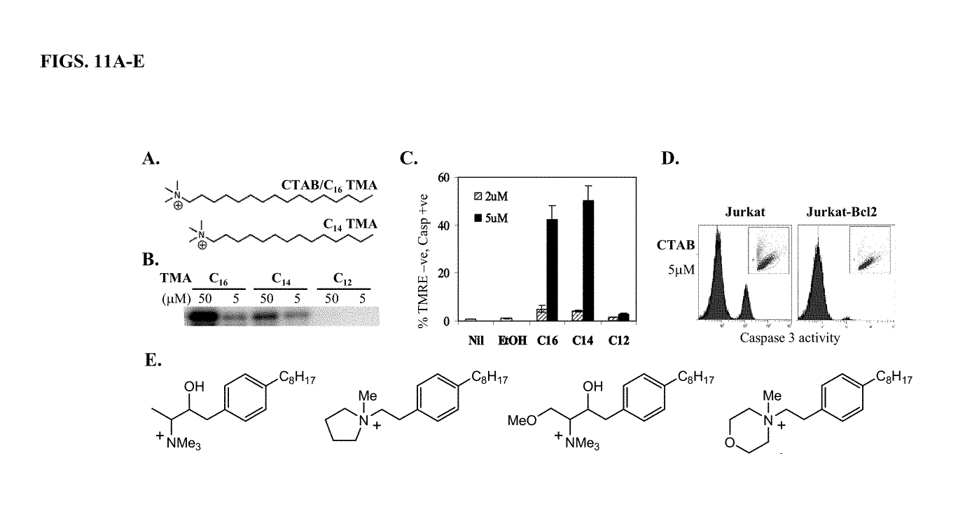

FIG. 12 depicts the amino acid sequences for protein 14-3-3 isoforms (SEQ ID NO:11), γ (SEQ ID NO:12), β (SEQ ID NO:13), η (SEQ ID NO:14), ε (SEQ ID NO:15), τ (SEQ ID NO:16) and σ (SEQ ID NO:17).

FIG. 13 depicts the residues defining sphingosine binding site R18/D020/D21 in monomer A together with R55/K85/E89 in monomer B of protein 14-3-3ζ. Alignments with the other protein 14-3-3 are also shown to indicate the corresponding residues.

FIG. 14 lists the residues of each of the protein 14-3-3 isoforms which correspond to R18, D20, D21, R55, S58, K85 and E89 of protein 14-3-3ζ. The numbering of the residues corresponds to the numbering of the residues as appears in SEQ ID NOs:11-17 and as further depicted in FIG. 12.

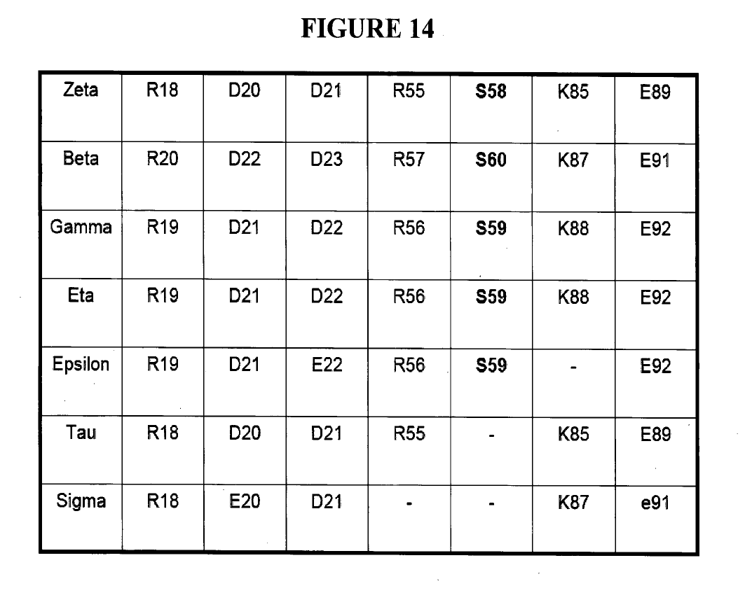

To the extent that some of these figures are printed in colour, these have been filed in colour and further copies of these figures in colour are also available on request to the applicant.

### DETAILED DESCRIPTION OF THE INVENTION

The present invention is predicated, in part, on the determination that 14-3-3 phosphorylation at Ser58 or analogous residue in the third helix is dependent on sphingosine binding to protein 14-3-3. Still further, the structural characteristics of both 14-3-3 and sphingosine which facilitate the subsequent phosphorylation of protein 14-3-3 have been identified. These findings, together with the development of an assay to screen for the functionality of a molecule to enable phosphorylation of protein 14-3-3, have facilitated the development of methods for regulating protein 14-3-3 phosphorylation and thereby regulating cellular functionality, such as apoptosis.

Accordingly, one aspect of the present invention provides a method of modulating protein 14-3-3 or homologue or variant functionality, said method comprising contacting said protein 14-3-3 with an agent which either modulates the interaction of sphingosine or homologue or variant with said protein 14-3-3 or which mimics the sphingosine interaction wherein agonising said interaction facilitates phosphorylation of Ser58 or analogous residue thereby inhibiting the functionality of said protein 14-3-3 and wherein antagonising said interaction inhibits phosphorylation of Ser58 or analogous residue thereby maintaining said protein 14-3-3 functionality.

In another aspect there is provided a method of modulating protein 14-3-3 or homologue or variant functionality in a mammal said method comprising administering to said mammal an effective amount of an agent for a time and under conditions sufficient to either modulate the interaction of sphingosine or homologue or variant with protein 14-3-3 or to mimic the sphingosine interaction wherein agonising said interaction facilitates phosphorylation of Ser58 or analogous residue thereby inhibiting the functionality of said protein 14-3-3 and wherein antagonising said interaction inhibits phosphorylation of Ser58 or analogous residue thereby maintaining said protein 14-3-3 functionality.

In still another aspect there is provided a method of modulating cellular functioning in a mammal said method comprising administering to said mammal an effective amount of an agent for a time and under conditions sufficient to modulate the interaction of sphingosine or homologue or variant with protein 14-3-3 or to mimic the sphingosine interaction wherein agonising said interaction facilitates phosphorylation of Ser58 or analogous residue thereby inhibiting the functionality of said protein 14-3-3 and wherein antagonising said interaction inhibits phosphorylation of Ser58 or analogous residue thereby maintaining said protein 14-3-3 functionality.

Reference to “protein 14-3-3” should be understood to include reference to all forms of protein 14-3-3. Without limiting the present invention to any one theory or mode of action, the 14-3-3 proteins are a conserved family of dimeric phospho-serine binding proteins that interact and modulate the functions of multiple cellular proteins and in so doing regulate many signalling pathways (Tzivion and Avruch 2002, J. Biol. Chem. 277:3061-64; Fu et al. 2000, Ann. Rev. Pharma. Tox. 40:617-47). The 14-3-3 proteins are composed of two 30 kDa monomer units that are each capable of binding a phospho-serine motif via an amphipathic groove. Dimers of 14-3-3 are formed by the N-terminal a helices, with helix 1 of one monomer interacting with helices 3 and 4 of another. Functionally, 14-3-3 proteins perform multiple roles in regulating cellular protein activities and importantly, these functions of 14-3-3 are dependent on its dimeric structure (Xing et al. 2000, supra; Yaffe 2002, FEBS Letts. 513:53-57), phosphorylation of Ser58 in helix 3 at the dimer interface of 14-3-3, results in conversion of dimeric 14-3-3 to a monomeric state (Woodcock et al. 2003, supra). Reference to “protein 14-3-3” should be understood to extend to any isoforms which arise from alternative splicing of protein 14-3-3 mRNA or allelic or polymorphic variants. To this end, the 14-3-3 proteins are a highly conserved family of seven phospho-serine binding proteins. The seven isoforms are the β (NCBI Ref. Sequence Number NM_003404.3; SEQ ID NO:13), ε (NCBI Ref. Sequence Number NM_006761.4; SEQ 1D NO:15), γ (NCBI Ref. Sequence Number NM_012479.3; SEQ ID NO:12), η (NCBI Ref. Sequence Number NM_003405.3; SEQ ID NO:14), σ (NCBI Ref. Sequence Number NM_006142.3; SEQ ID NO:17), τ (NCBI Ref. Sequence Number NM_006826.2; SEQ ID NO:16) and ζ (NCBI Ref. Sequence Number NM_003406.3; SEQ ID NO: 11) forms.

Reference to “variants” should be understood to extend to functional mutants. Reference to “homologues” should be understood as a reference to 14-3-3 proteins from species other than human. Reference to a “functional” 14-3-3 protein should be understood as a reference to a molecule which can undergo phosphorylation thereby leading to monomer formation. It should be understood that “protein 14-3-3” is also interchangeably referred to as “14-3-3” in this specification. Both terms should be understood as a reference to the same molecule. In one embodiment, said protein 14-3-3 is human 14-3-3.

In another embodiment, said human protein 14-3-3 is the ζ form.

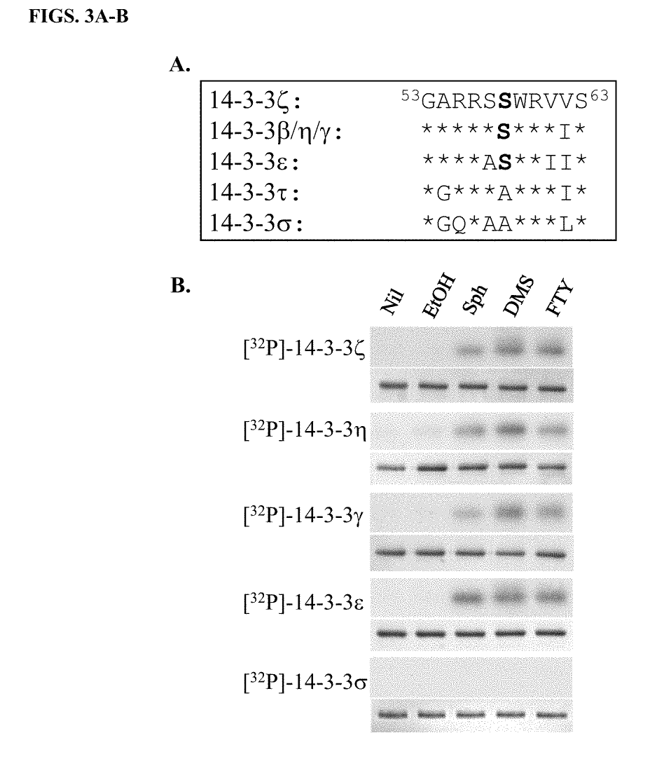

Reference to “Ser58” should be understood as a reference to the serine residue at amino acid position 58 in the third helix of protein 14-3-3ζ. This residue is depicted in the SEQ ID NO:1 motif. Reference to “analogous residue” should be understood as a reference to the corresponding serine residue of other protein 14-3-3 isoforms. Examples of corresponding serine residues are provided in the context of protein 14-3-3β, η, γ and ε and are depicted in FIG. 3. To this end, the serine residue in issue corresponds to residue 59 in the context of 14-3-3η and 14.-3-3γ and is found within the motif GARRSSWRVIS which is numbered as residues 54-64 of the 14-3-3η and γ sequence; In the context of 14-33ε, this serine residue is numbered 59 within the context of the motif GARRASWRIIS, which is itself numbered as residues 54-64 of the 14-3-3ε sequence. With respect to 14-3-3β, the subject serine residue is numbered 60 within the context of the motif GARRSSWRVIS, which is itself numbered as residues 55-65 of the 14-3-3β sequence.

Accordingly, in one embodiment said serine 58 or analogous residue is selected from the list consisting essentially of:

(i) Ser58 of protein 14-3-3ζ;

(ii) Ser59 of protein 14-3-3η;

(iii) Ser59 of protein 14-3-3γ;

(iv) Ser59 of protein 14-3-3ε; and

(v) Ser60 of protein 14-3-3β.

Reference to “protein 14-3-3 functionality” should be understood as a reference to any one or more cellular activities which this protein modulates. Without limiting the present invention to any one theory or mode of action, this family of phospho-serine binding proteins are involved in the regulation of cell division, growth and apoptosis. The 14-3-3 proteins influence the function of bound phospho-serine proteins via a variety of mechanisms including sequestering them from cellular targets, controlling their enzymatic activity, relocating them or acting as adaptor molecules in mediating the association of two distinct client proteins. As detailed hereinbefore, the 14-3-3 proteins are themselves regulated by phosphorylation.

Phosphorylation of the Ser58 or analogous residue of the 14-3-3 protein disrupts dimer formation, converting it to the monomer structure, which reduces its functionality.

In one embodiment the cellular functioning which is the subject of modulation is cell division, cell growth or cellular apoptosis.

Accordingly, there is provided a method of modulating cellular apoptosis in a mammal said method comprising administering to said mammal an effective amount of an agent for a time and under conditions sufficient to either modulate the interaction of sphingosine or homologue or variant with protein 14-3-3 or mimic the interaction of sphingosine or homologue or variant with protein 14-3-3 wherein agonising said interaction facilitates phosphorylation of Ser58 or analogous residue thereby inducing apoptosis and wherein antagonising said interaction inhibits phosphorylation of Ser58 or analogous residue thereby inhibiting apoptosis.

Byregulating cellular apoptosis, overall viable cell numbers can be controlled thereby effectively providing a means of regulating the cellular division or proliferation.

Reference to “phosphorylation” should be understood as a reference to the addition of a phosphate group to the hydroxyl groups on proteins, in particular to the side chains of the amino acids serine, threonine or tyrosine. Without limiting the present invention to any one theory or mode of action, this process of phosphorylating proteins is normally catalysed by a protein kinase (such molecules herein being referred to as “phosphorylation catalysts”), often a specific protein kinase, with ATP acting as the phosphate donor. The phosphorylation of proteins is generally found to regulate the activity Of the subject protein.

As detailed hereinbefore, the phosphorylation of a protein is an event which occurs in the context of specific amino acid residues of a subject protein. In this regard, Ser58 or analogous residue is the amino acid residue which is relevant to phosphorylation of the human protein 14-3-3.

As detailed hereinbefore, the binding of sphingosine to protein 14-3-3 enables the phosphorylation of Ser58 or analogous residue of protein 14-3-3. Reference to “sphingosine” should be understood to have a corresponding meaning to that provided for “protein 14-3-3”. That is, “sphingosine” includes reference to all forms of sphingosine and to functional homologues and variants thereof. Without limiting the present invention to any one theory or mode of action, sphingosine is a long-chain amino alcohol that bears similarity to glycerol with a quaternary ammonium group on the 2-carbon and a hydrophobic chain attached to the 3-carbon. Sphingosine forms the class of sphingolipids when it carries an acyl group joined by an amide link to the nitrogen. It forms sphingomyelin when phosphoryl choline is attached to the 1-hydroxyl group and gives rise to the cerebroside and ganglioside classes of glycolipids when oligosaccharides are attached to the 1-hydroxyl group.

Reference to “modulating” either protein 14-3-3 functionality or the interaction of sphingosine with protein 14-3-3 should be understood as a reference to upregulating or downregulating the subject functional activity or interaction. Reference to upregulating or downregulating in this regard should be understood to include both increasing or decreasing the level, degree or rate at which the functional activity or interaction event occurs, in addition to including reference to inducing, ablating or maintaining the subject functional activity or interaction event.

Reference to “mimics the sphingosine interaction” should be understood as a reference to the use of an agent which interacts with protein 14-3-3 and facilitates the phosphorylation of Ser58 or analogous residue of protein 14-3-3. Accordingly, the agent which is utilised in accordance with the method of the present invention may be an agent which induces the activity/event (e.g. by itself mimicking the sphingosine functionality), agonises an activity or event which has already undergone onset, antagonises a pre-existing activity or event or entirely ablates or prevents such an activity or event.

As detailed hereinbefore, the present invention is predicated on the determination that the interaction of sphingosine with protein 14-3-3 renders protein 14-3-3 phosphorylatable. In turn, this causes the 14-3-3 protein to dissociate to the monomer structure which, inter alia, can no longer function to protect a cell from apoptotic signals such as BAD and ASK-1.

Accordingly, reference to the use of an agent which agonises the interaction of sphingosine with protein 14-3-3 should be understood as a reference to the use of an agent which either mimics sphingosine in that it binds to protein 14-3-3 and enables phosphorylation of Ser58 or analogous residue or which induces or otherwise facilitates the interaction of intracellular sphingosine with protein 14-3-3. Conversely, an agent which antagonises the interaction of sphingosine with protein 14-3-3 should be understood as a reference to an agent which either prevents the interaction of sphingosine with protein 14-3-3 or which antagonises an existing interaction of sphingosine with protein 14-3-3 such that it is ineffective or less effective.

It should be understood that modulation of the interaction between sphingosine and protein 14-3-3 (either in the sense of up-regulation or down-regulation) may be partial or complete. Partial modulation occurs where only some of the sphingosine/protein 14-3-3 interactions which may normally occur in a given cell are affected by the method of the present invention (for example, the agent which is contacted with the subject cell is provided at a concentration insufficient to saturate the intracellular sphingosine/protein 14-3-3 interactions) while complete modulation occurs where all sphingosine/protein 14-3-3 interactions are modulated.

Modulation of the interaction between sphingosine and protein 14-3-3 or mimicking the sphingosine interaction may be achieved by any one of a number of techniques including, but not limited to:
(i) introducing into a cell a molecule which mimics sphingosine or derivative, homologue, analogue or mimetic thereof or introducing the proteinaceous form of sphingosine or derivative, analogue, homologue or mimetic thereof in order to modulate the intracellular concentration of sphingosine which is available.
(ii) introducing into a cell a proteinaceous or non-proteinaceous molecule which antagonises the interaction between protein 14-3-3 and sphingosine, such as a competitive inhibiter or antibody.
(iii) introducing into a cell a proteinaceous or non-proteinaceous molecule which agonises the interaction between. protein 14-3-3 and sphingosine.

Reference to “agent” should be understood as a reference to any proteinaceous or non-proteinaceous molecule which modulates or mimics the interaction of sphingosine with protein 14-3-3 and includes, for example, the molecules detailed in points (i)-(iii), above. The subject agent may be linked, bound or otherwise associated with any proteinaceous or non-proteinaceous molecule. For example, it may be associated with a molecule which permits its targeting to a localised region. In one embodiment, the subject agent is sphingosine itself, or functional derivative, analogue, homologue or mimetic thereof, which is introduced to facilitate phosphorylation of protein 14-3-3.

Said proteinaceous molecule may be derived from natural, recombinant or synthetic sources including fusion proteins or following, for example, natural product screening. Said non-proteinaceous molecule may be derived from natural sources, such as for example natural product screening, or may be chemically synthesised. For example, the present invention contemplates chemical analogues of sphingosine capable of acting as agonists or antagonists of the sphingosine interaction. Chemical agonists may not necessarily be derived from sphingosine but may share certain conformational similarities. Alternatively, chemical agonists may be specifically designed to mimic or upregulate certain physiochemical properties of sphingosine. Antagonists may be any compound capable of blocking, inhibiting or otherwise preventing sphingosine and protein 14-3-3 from interacting. Antagonists include antibodies (such as monoclonal and polyclonal antibodies) specific for sphingosine or protein 14-3-3, or parts of said sphingosine or protein .14-3-3. Reference to antagonists also includes antigens which competitively inhibit sphingosine/protein 14-3-3 interaction, siRNA, miRNA, antisense molecules (eg. antisense RNA), ribozymes, DNAzymes, aptamers (eg. RNA aptamers), or molecules suitable for use in co-suppression. The proteinaceous and non-proteinaceous molecules referred to in points (i)-(iii), above, are herein collectively referred to as “modulatory agents”.

Screening for modulatory agents may be performed by any suitable method. In one embodiment, the findings of the present invention have enabled the development of a screening assay which identifies agents which either agonise (e.g. mimic) or antagonise the binding of sphingosine to protein 14-3-3 to reveal Ser58 or analogous residue and enable its phosphorylation. This assay is based on the surprising determination that it is not exposure of protein 14-3-3 to an activated kinase which effects 14-3-3 phosphorylation but, rather, the binding of sphingosine to 14-3-3 which effects a structural change to the 14-3-3 dimer. This structural change exposes Ser58 or analogous residue thereby rendering it phosphorylatable.

Accordingly, in a related aspect there is provided a method for detecting an agent which facilitates the phosphorylation of protein 14-3-3 at Ser58 or analogous residue said method comprising contacting a putative agent with protein 14-3-3 and a serine kinase or catalytic subunit thereof and screening for protein 14-3-3 phosphorylation at Ser58 or analogous residue.

In a related aspect there is provided a method for detecting an agent which antagonises the phosphorylation of protein 14-3-3 at Ser58 or analogous residue said method comprising:
(i) contacting a putative agent with protein 14-3-3 and sphingosine or functional equivalent;
(ii) contacting the composition of (i) with a serine kinase or catalytic subunit thereof; and screening for protein 14-3-3 phosphorylation at Ser58 or analogous residue wherein a reduction in the level of phosphorylation relative to a control level is indicative of the antagonistic activity of said putative agent.

In one embodiment said serine 58 or analogous residue is selected from the list consisting essentially of:

(i) Ser58 of protein 14-3-3ζ;

(ii) Ser59 of protein 14-3-3η;

(iii) Ser59 of protein 14-3-3γ;

(iv) Ser59 of protein 14-3-3ε; and

(v) Ser60 of protein 14-3-3β.

Said putative agent may be any proteinaceous or non-proteinaceous molecule of interest and may take any suitable form. For example, proteinaceous agents may be glycosylated or unglycosylated, phosphorylated or dephosphorylated to various degrees and/or may contain a range of other molecules fused, linked, bound or otherwise associated with the proteins such as amino acids, lipids, carbohydrates or other peptides, polypeptides or proteins. Similarly, the subject non-proteinaceous molecules may also take any suitable form. Both the proteinaceous and non-proteinaceous agents herein described may be linked, bound otherwise associated with any other proteinaceous or non-proteinaceous molecules. For example, in one embodiment of the present invention said agent is associated with a molecule which permits its targeting to a . localised region, such as a specific tissue.

Reference to “mimetics” or “mimics” of sphingosine should be understood as molecules exhibiting any one or more of the functional activities of sphingosine, which mimetics or mimics may be derived from any source such as being chemically synthesised or identified via screening processes such as natural product screening. For example, chemical or functional equivalents can be designed and/or identified utilising well known methods such as combinatorial chemistry or high throughput screening of recombinant libraries or following natural product screening. These methods may also be utilised to design any of the modulatory agents which are useful in the method of the present invention.

Reference to “serine kinase” should be understood as a reference to any protein kinase which is capable of phosphorylating serine, in particular Ser58 or analogous residue of protein 14-3-3, or catalytic subunit thereof. Examples of serine kinases suitable for use in the method of the invention include, but are not limited to, protein kinase A (Ma et al. 2005; Gu et al. 2006), PKCδ (Hamaguchi et al. 2003), other PKC's (Jones et al. 1995), AKT/PKB (Powell et al. 2002), MAPKAP-2 (Powell et al. 2003), SOK-1/YSK-1 (Preisinger et al. 2004). Reference to “serine kinase” should also be understood to extend to any isoforms which arise from alternative splicing of the serine kinase mRNA or allelic or polymorphic variants. Reference to “homologues” and “variants” should be understood to have an equivalent meaning as hereinbefore provided. In one embodiment, said serine kinase is protein kinase A.

Accordingly, in one embodiment there is provided a method for detecting an agent which facilitates the phosphorylation of protein 14-3-3 at Ser 58 or analogous residue said method comprising contacting a putative agent with protein 14-3-3 and protein kinase A or catalytic subunit thereof and screening for protein 14-3-3 phosphorylation at Ser58 or analogous residue.

In another embodiment there is provided a method for detecting an agent which antagonises the phosphorylation of protein 14-3-3 at Ser58 or analogous residue said method comprising:
(i) contacting a putative agent with protein 14-3-3 and sphingosine or functional equivalent;
(ii) contacting the composition of (i) with protein kinase A or catalytic subunit thereof; and screening one or both of for protein 14-3-3 phosphorylation at Ser58 or analogous residue or protein 14-3-3 ubiquitination wherein a reduction in the level of phosphorylation relative to a control level is indicative of the antagonistic activity of said putative agent.

Reference to “protein 14-3-3” and “sphingosine” should be understood to have a corresponding meaning as hereinbefore provided. A “functional equivalent” is a reference to any other molecule which one could substitute instead of sphingosine but which would produce the same results.

Reference to detecting an agent which “facilitates” the phosphorylation of protein 14-3-3 should be understood as a reference to an agent, the presence of which in the reaction mixture leads to phosphorylation of Ser58 or analogous residue. As detailed hereinbefore, this agent functions by either agonising the interaction of sphingosine with protein 14-3-3 or by mimicking sphingosine to directly interact with protein 14-3-3 and enable phosphorylation of Ser58 or analogous residue by the serine kinase.

In the context of screening for an agonist or mimic of the sphingosine/protein 14-3-3 interaction, one screens for the occurrence of phosphorylation. The level of phosphorylation which occurs will depend on the relative amount and activity of the putative agent. In one embodiment, one is screening for an agent which functions as a sphingosine mimetic, such as a sphingosine analogue.

In accordance with this embodiment there is provided a method of detecting a sphingosine mimetic, said method comprising contacting a putative mimetic with protein 14-3-3 and protein kinase A or catalytic subunit thereof and screening for protein 14-3-3 phosphorylation at Ser58 or analogous residue.

In one embodiment said serine 58 or analogous residue is selected from the list consisting essentially on
(i) Ser58 of protein 14-3-3ζ;
(ii) Ser59 of protein 14-3-3η;
(iii) Ser59 of protein 14-3-3γ;
(iv) Ser59 of protein 14-3-3ε; and
(v) Ser60 of protein 14-3-3β.

Accordingly, in the context of this aspect of the present invention, one is screening for the occurrence of any level of phosphorylation. Depending on the extent of the phosphorylation, which can be measured either qualitatively or quantitatively, this will provide iriformation in relation to the level of activity of the putative agent relative to the level of activity of endogenous sphingosine.

In the context of screening for an antagonist of the sphingosine/protein 14-3-3 interaction, one assesses the level of phosphorylation which occurs in the test assay relative to that which occurs in a control test, with any decrease in the level of phosphorylation in the test relative to the control indicating the presence of an antagonist. The result of the control test is referred to as the “control level”. Accordingly, reference to a “control level” should be understood as a reference to the level of protein 14-3-3 phosphorylation in an assay which corresponds to the test assay in terms of the assay parameter, other than the fact that there is no antagonist present in the control assay which would competitively inhibit the binding of sphingosine. It should be understood that the control levels may be determined de novo with each testing of a putative agent or a standard set of test parameters can be established such that a standard set of results can be established (e.g. a standard curve) for ongoing use in the context of qualitative or quantitative analysis.

Reference to detecting the “level” of phosphorylation should be understood as a reference to either qualitatively and/or quantitatively assessing the amount of phosphorylation. At its simplest, assessment by eye of the intensity of a band which has developed, such as on an autoradiograph following incorporation of a radioactive phosphate (e.g. [³²P]-γATP) or immunoblotting using an antibody that specifically recognises the phosphorylated residue, in isolation (i.e. the presence/absence of any phosphorylation) or relative to a control test may be performed, wherein a darker and/or thicker band is indicative of a higher level of phosphorylation than a fainter and/or thinner band. A corresponding type of analysis can be qualitatively or quantitatively performed with reporter readouts. More sophisticated analysis can be performed utilising equipment such as a densitometer based on visible light or fluorescence, which can empirically calculate the concentration of phosphorylated 14-3-3 in a given band relative to a standard.

“Contacting” the individual components of these assays can be performed by any suitable method. For example, in the context of the agonist assay, the agent, protein 14-3-3 and serine kinase can be contacted simultaneously by adding all three components to a test vessel simultaneously. Alternatively, the agent and protein 14-3-3 may be initially contacted and binding facilitated, with the serine kinase thereafter introduced to the test. With respect to screening for an antagonist, the assay can be designed as a competitive inhibition assay or else the agent and 14-3-3 can undergo a preliminary contact step, such that all binding of the agent, if any, is saturated and thereafter sphingosine can be introduced. To this end, the design of the assay to screen for protein 14-3-3 phosphorylation can be pursued in any one of a variety of means including, but not limited to:

(i) SDS-PAGE analysis after [³²P]-γATP incorporation

(ii) P81 paper scintillation counting after radiolabel incorporation;

(iii) immunoblotting using a phospho-Ser58 antibody;

(iv) scintillation proximity assay after [³³P]-γATP incorporation; or

(v) ELISA using a phospho-Ser58 antibody.

Reference to “interaction” should be understood as a reference to any form of interaction including the formation of covalent bonds, hydrogen bonds, Van Der Waals forces or any other mechanism of interaction.

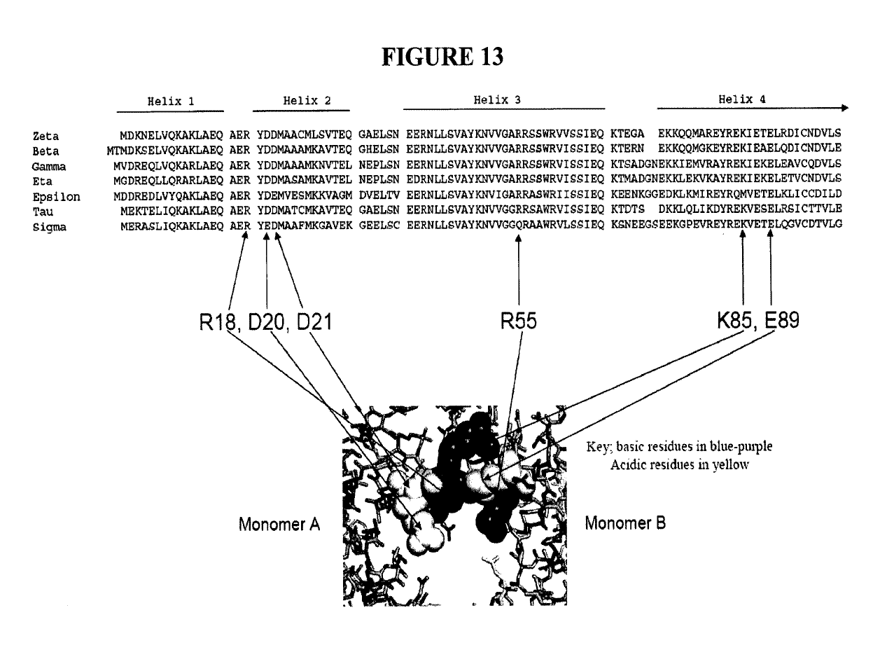

In a related aspect, the inventors have identified the dimer interface of protein 14-3-3 into which sphingosine binds in order to effect conformational alteration of protein 14-3-3 to thereby enable phosphorylation of Ser58 or analogous residue. That is, accessibility of the phosphorylation site is effected. Without limiting the present.invention to any one theory or mode of action, it is the basic nature of sphingosine which is relevant to its effect. To this end, three exposed acidic residues of protein 14-3-3, being D20, D21 and E89, have been determined to exhibit functional importance in terms of the modulation of protein 14-3-3ζ phosphorylatability in response to lipid. These acidic residues are conserved across the protein 14-3-3 family and cluster at the base of the aperture on the underside of the protein with respect to the phospho-serine peptide binding groove (FIG. 13). The dimer interface of the 14-3-3 protein lies at the bottom of the phospho-serine binding groove and comprises a central hole of about 10 Å in diameter in which the Ser58 or analogous residue phosphorylation site is positioned.

Still without limiting the present invention in any way, and in the context of protein 14-3ζ, the D20 residue is involved with the sphingolipid, its exposed position in the aperture being consistent with this. D21 and E89 function to maintain dimer formation through salt bridge formation with their salt bridge partners, K85 and R18, respectively, of the opposite 14-3-3 polypeptide which forms the dimer. By interacting a molecule with D20, the interaction of D21 and E89 with their salt bridge binding partners is weakened thereby exposing the protein 14-3-3 dimer interface and enabling Ser58 phosphorylation. Even in the absence of interaction directly with D20, breakage of the salt bridge of one or both of D21 and E89 (or the reciprocal residues K85 and R18) enables sufficient conformational change to effect Ser58 phosphorylation. A corresponding outcome is achieved by disrupting the salt bridge which forms between E5 and K74. R55 is a small molecule binding site within this cleft.

In terms of identifying agents suitable for interacting with this cleft and thereby mimicking sphingosine functionality, assays based on screening for functional readouts are hereinbefore described. However, based on the identification of the structural features of the cleft into which sphingosine binds and, further, the specific residues which are relevant to maintaining salt bridge formation, and thereby the dimer structure, there has been enabled the rational design of agents which effectively act as sphingosine mimetics by specifically targeting these residues and the cleft as a means for inducing conformatorial change and thereby phosphorylation of Ser58. In light of the availability of the 14-3-3 dimer crystal structure, such molecules can be rationally designed in silica as a matter of routine procedure and thereafter routinely synthetically generated for testing in the functional assays herein described.

Accordingly, in one embodiment said agent which agonises the interaction of sphingosine with protein 14-3-3 is a sphingosine mimetic, which agent interacts with one or more of protein 14-3-3 residues D20, D21, E89, K85, R18, E5, K74 or R55 or analogous residue. More specific examples of such agents are provided hereafter in the context of the class of agents represented by formula (1).

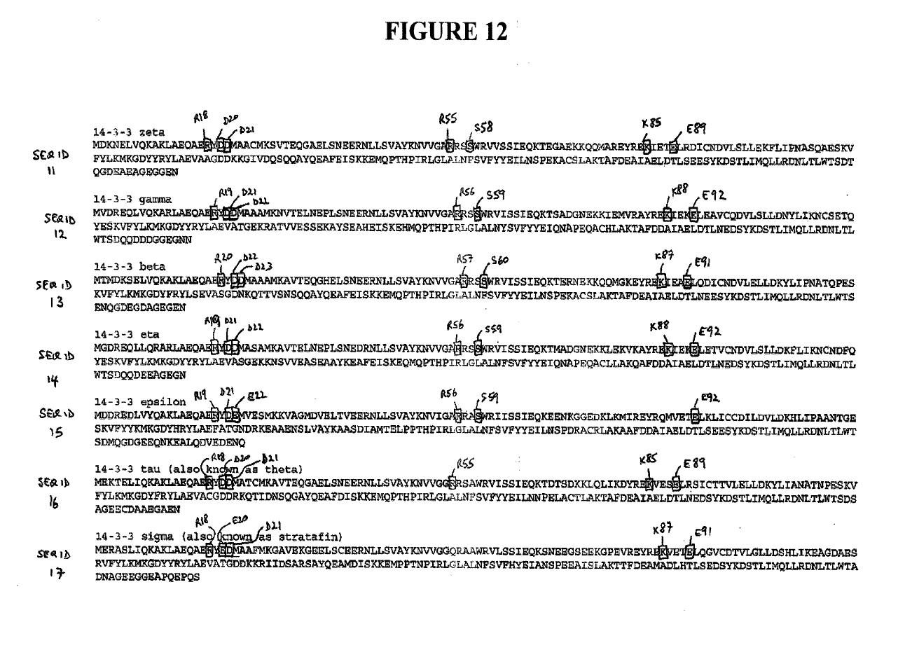

As detailed hereinbefore, residues D20, D21, E89, R18, K85, E5, K74 and R55 correspond to the residue numbering of protein 14-3-3ζ (SEQ 1D NO:1 1). However, the analogous residues of the other protein 14-3-3 isoforms are routinely identifiable based on the knowledge of all of these highly homologous sequences (see FIGS. 12 and 13).

Specifically, said analogous protein 14-3-3ζ residues R18, D20, D21, R55, R58, K85 and E89 (SEQ ID NO:11) are, respectively, as follows:

(i) R19, D21, D22, R56, S59, K88 and E92 of protein 14-3-3γ (SEQ ID NO:12);

(ii) R20, D22, D23, R57, S60, 1(87 and E91 of protein 14-3-3β (SEQ ID NO:13);

(iii) R19, D21, D22, R56, S59, K88 and E92 of protein 14-3-3η (SEQ ID NO:14);

(iv) R19, D21, E22, R56, S59 and E92 of protein 14-3-3ε (SEQ ID NO:15);

(v) R18, D20, D21, R55, K85 and E89 of protein 14-3-3τ (SEQ ID NO:16);

(vi) R18, E20, D21, K87 and E91 of protein 14-3-3σ (SEQ ID NO:17).

Accordingly, in one embodiment said protein 14-3-3 is 14-3-3ζ and said residues are D20, D21, E89. K85, R18, E5, K74 or R55.

In another embodiment, said protein 14-3-3 is selected from:

(i) protein 14-3-3γ and said residues are R19, D21, D22, R56, K88 or E92;

(ii) protein 14-3-3β and said residues are R20, D22, D23, R57, K87 or E91;

(iii) protein 14-3-3η and said residues are R19, D21, D22, R56, K88 or E92;

(iv) protein 14-3-3ε and said residues are R19, D21, E22, R56 or E92;

(v) protein 14-3-3τ and said residues are R18, D20, D21, R55, K85 or E89;

(vi) protein 14-3-3σ and said residues are R18, E20, D21, K87 or E91.

In yet another embodiment, said residues are D20, D21, E89, K85 or R18.

In still another embodiment, said agent is non-phosphorylated.

In yet another embodiment of the present invention, the assay hereinbefore described has been performed to identify agonist molecules and sphingosine mimetics. To this end, reference to “agonist” should be understood to include reference to sphingosine mimetics. In accordance with these findings, said agent may be represented by formula (I):

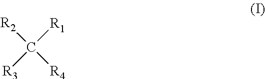

R₁ represents a saturated or unsaturated, branched or linear optionally substituted C₆-C₂₈aliphatic group:
R₂ represents a C₂-C₄ alkyl substituted from 1 to 3 times with groups independently selected from
   (CH₂)ₘOR′ where R′ is H or C₁₋₅ alkyl, —(CH₂)ₘCOOR″ where R″ is H or C₁₋₅ alkyl, —(CH₂)ₘNH₂, —(CH₂)ₘNH₃, —(CH₂)ₘN(C₁₋₆alkyl)₂, —(CH₂)ₘCHNH(COR′″)—(CH₂)ₚOH, —(CH₂)ₘN(C₁₋₆alkyl)₃, —(CH₂)ₘP(O)(OH)₂, —(CH)(—NHC(O)—C₁-C₂₈alkyl)-(CH₂)ₚ—O—P(O)(OH)—O—(CH₂)ₘ—NH₃, and —(CH)(—NHC(O)—C₁-C₂₈alkyl)-(CH₂)ₚ—O—P(O)(OH)—O—(CH₂)ₘ—N(C₁-C₄alkyl)₃;

R₃ and R₄ independently represent H or —(CH₂)ₙOR′″ where R′″ is H or C₁₋₃ alkyl;

m is 0, 1, or 2;

n is 0, 1, or

p is 1 or 2;

or a pharmaceutically acceptable salt thereof.

In an embodiment R₁ represents a C₆-C₂₈ aliphatic chain derived from the following fatty acids:
saturated fatty acids: (e.g., caproic, caprylic, perlargonic, capric, lauric, myristic, palmitic, daturic, steric, arachidic, behenic, lignoceric, cerotic, carboceric and montanic acid),
mono or multi-branched-chain fatty acids: (e.g., phytomonic, laetiporic, mycoceranic, mycocerosic, phthioceranic, pristanic, and retinoic acid),
branched methoxy fatty acids: (e.g., 2-methoxy-14-methylpentadecanoic acid),
cis and trans monoenoic fatty acids: (e.g., caproleic, lauroleic, linderic, sapienic, petroselenic, oleic, elaidic, vaccenic, gadoleic, ondoic, cetoleic, erucic, nervonic, and t3-hexadecanoic acid),
ring containing fatty acids: (e.g., lactobaeillic, majusculoic, gorlic, hydnocarpic, chaulmoogric, 11-cyclohexylundecanoic, 13-cyclohexyltridecanoic, 10,13-epoxy-11-methyloctadeca-10,12-dienoic, and lipoic acid),
acetylenic and polyacetylenic fatty acids: (e.g., tariric, santalbic, 6,9-octadecenynoic, crepenynic, scleropyric, phomallenic, and oropheic acid),
polyenoic fatty acids: (e.g., linoleic, arachidonic, and nisinic acid),
hydroxyl, thio, halo, nitro, arseno, and phosphorus containing fatty acids: (e.g., 19-fluoro-oleic, 9-chloro-10-hydroxypalmitic, and 12-nitro-9-cis, 12-cis-octadecadienoate acid).

Accordingly, it will be appreciated that in the case of the C₆-C₂₈ aliphatic group which is representative of R₁, the term “optionally substituted” indicates that one or more saturated carbon atoms may be substituted for a heteroatom or heterogroup, such as O, S, NH, C(O), SO₂ or cyclic group (including arylene (e.g., phenylene), cyclohexylene, heteroarylene, etc). For example a substituted C₆-C₂₈ aliphatic group could be represented by a group such as

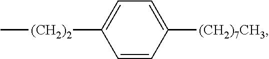

—(CH₂)₂—NH—(CH₂)₄—NH(CH₂)₃—NH₂, —(CH₂)₂₀(CH₂)₂O(CH₂)₁₅CH₃, etc and the like. The term also denotes that the substituent may also be pendant to the aliphatic chain such as —(CH₂)₁₅CH(OH)—CH₃, —(CH₂)₂₀CH(CF₃)—CH₃ and the like. Preferred pendant substituent groups include, halo (e.g., Br, Cl, F), hydroxyl, carboxyl, nitro, cyano, —C(O)O(C₁-C₃alkyl), —C(O)(C₁₋₃alkyl), and trihalo methyl.

In an embodiment R₃ is OH and R₄ is H.

In an embodiment both R₃ and R₄ are H.

In an embodiment R₃ is OH and R₄ is H and R₂ is selected from:

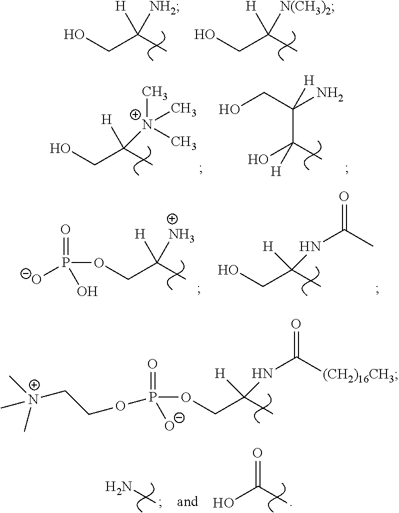

In an embodiment R₁ represents a saturated or unsaturated linear substituted C₁₂-C₂₈ alkyl, R₂ represents —(CH₂)ₘNH₃, —(CH₂)ₘN(C₁₋₆ alkyl)₃, m is 0, and R₃ and R₄ represent H. Accordingly, it will be appreciated that in an embodiment the agent may be a quartenary ammonium compound. Examples of such compounds include known cationic surfactant molecules, for example, hexadecyl-trimethyl ammonium (‘C16-TMA’) (and halide salt forms thereof such as CTAB and CTAC).

Specific examples of possible agents of the present invention include:

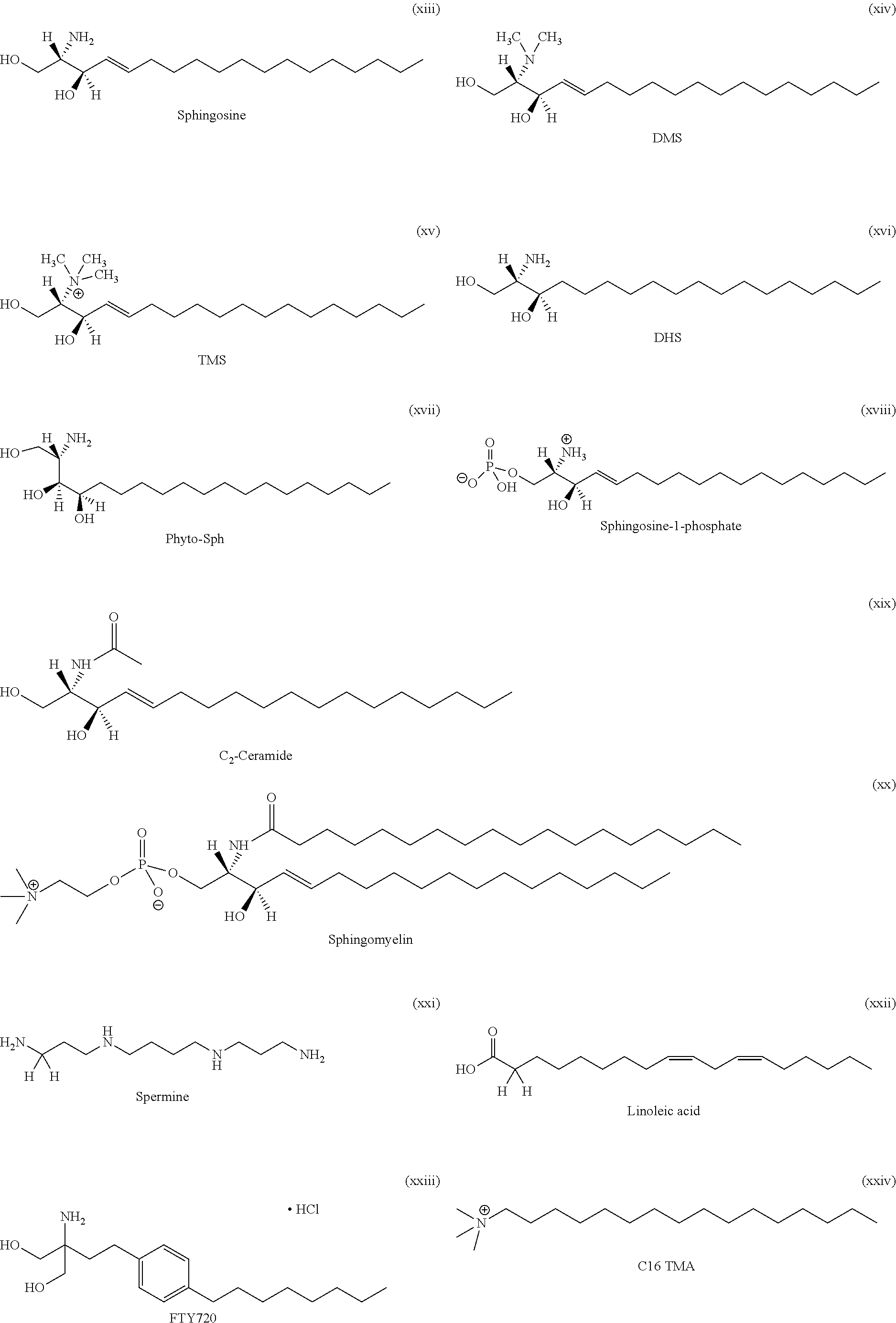

The agents of the present invention may also be presented as pharmaceutically acceptable salts thereof. Suitable pharmaceutically acceptable salts include, but are not limited to salts of pharmaceutically acceptable inorganic acids such as hydrochloric, sulphuric, phosphoric, nitric, carbonic, boric, sulfamic, and hydrobromic acids, or salts of pharmaceutically acceptable organic acids such as acetic, propionic, butyric, tartaric, maleic, hydroxymaleic, fumaric, maleic, citric, lactic, mucic, gluconic, benzoic, succinic, oxalic, phenylacetic, methanesulphonic, toluenesulphonic, benezenesulphonic, salicyclic sulphanilic, aspartic, glutamic, edetic, stearic, palmitic, oleic, lauric, pantothenic, tannic, ascorbic and valeric acids.

Base salts include, but are not limited to, those formed with pharmaceutically acceptable cations, such as sodium, potassium, lithium, calcium, magnesium, ammonium and alkylammonium. In particular, the present invention includes within its scope cationic salts eg sodium or potassium salts, or alkyl esters (eg methyl, ethyl) of the phosphate group.

Basic nitrogen-containing groups may be quarternised with such agents as lower alkyl halide, such as methyl, ethyl, propyl, and butyl chlorides, bromides and iodides; dialkyl sulfates like dimethyl and diethyl sulfate; and others.

It will also be recognised that the agents of the invention may possess asymmetric centres and are therefore capable of existing in more than one stereoisomeric form. For instance, where each of R₁—R₄ is a different chemical group the agents of formula (I) will be chiral. The invention thus also relates to agents in substantially pure isomeric form at one (or more) asymmetric centres eg., greater than about 90% ee, such as about 95% or 97% ee or greater than 99% ee, as well as mixtures, including racemic mixtures, thereof. Such isomers may be prepared by asymmetric synthesis, for example using chiral intermediates (such as from chiral fatty acid esters), or mixtures may be resolved by conventional methods, eg., chromatography, or use of a resolving agent.

In yet another embodiment, it has been determined that said agent is a non-acylated basic sphingolipid which is not phosphorylated. By “non-acylated” is meant that it is not conjugated to a hydrocarbon chain at the primary amine. Examples of such agents include sphingosine, DHS, TMS, Phyto-Sph as illustrates above. Without limiting the present invention to any one theory or mode of action, the lipid modulation of protein 14-3-3 occurs with lipids carrying a net positive charge, indicating that basic sphingosine interacts directly with the acidic 14-3-3 substrate.

Accordingly, in one embodiment there is provided a method of inhibiting protein 14-3-3 or homologue or variant functioning said method comprising contacting said protein 14-3-3 with an agent of formula (I):

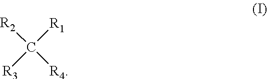

R₁ represents a saturated or unsaturated, branched or linear optionally substituted C₆-C₂₈aliphatic group;
R₂ represents a C₂-C₄ alkyl substituted from 1 to 3 times with groups independently selected from
   —(CH₂)ₘOR′ where R′ is H or C₁₋₅ alkyl, —(CH₂)ₘCOOR″ where R″ is H or C₁₋₅ alkyl, —(CH₂)ₘNH₂, —(CH₂)ₘNH₃, —(CH₂)ₘN(C₁₋₆alkyl)₂, —(CH₂)ₘCHNH(COR′″)—(CH₂)ₚOH, —(CH₂)ₘN(C₁₋₆alkyl)₃, —(CH₂)ₘP(O)(OH)₂, —(CH)(—NHC(O)—C₁-C₂₈alkyl)-(CH₂)ₚ—O—P(O)(OH)—O—(CH₂)ₘ—NH₃, and —(CH)(—NHC(O)—C₁-C₂₈alkyl)-(CH₂)ₚ—O—P(O)(OH)—O—(CH₂)ₘ—N(C₁-C₄alkyl)₃;
R₃ and R₄ independently represent H or —(CH₂)ₙOR′″ where R′″ is H or C₁₋₃ alkyl;

m is 0, 1, or 2;

n is 0, 1, or 2;

p is 1 or 2;

or a pharmaceutically acceptable salt thereof.

In another embodiment, there is provided a method of inhibiting protein 14-3-3 or homologue or variant functionality in a mammal said method comprising administering to said mammal an effective amount of an agent of formula (I):

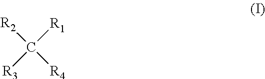

R₁ represents a saturated or unsaturated, branched or linear optionally substituted C₆-C₂₈aliphatic group;
R₂ represents a C₂-C₄ alkyl substituted from 1 to 3 times with groups independently selected from
   —(CH₂)ₘOR′ where R′ is H or C₁₋₅ alkyl, —(CH₂)ₘCOOR″ where R″ is H or C₁₋₅ alkyl, —(CH₂)ₘNH₂, —(CH₂)ₘNH₃, —(CH₂)ₘN(C₁₋₆alkyl)₂, —(CH₂)ₘCHNH(COR′″)—(CH₂)ₚOH, —(CH₂)ₘN(C₁₋₆alkyl)₃, —(CH₂)ₘP(O)(OH)₂, —(CH)(—NHC(O)—C₁-C₂₈alkyl)-(CH₂)ₚ—O—P(O)(OH)—O—(CH₂)ₘ—NH₃, and —(CH)(—NHC(O)—C₁-C₂₈alkyl)-(CH₂)ₚ—O—P(O)(OH)—O—(CH₂)ₘ—N(C ₁-C₄alkyl)₃;
R₃ and R₄ independently represent H or —(CH₂)ₙ OR′″ where R′″ is H or C₁₋₃ alkyl;

m is 0, 1, or 2;

n is 0, 1, or 2;

p is 1 or 2;

or a pharmaceutically acceptable salt thereof.

In yet still another embodiment, there is provided a method of downregulating cellular apoptosis in a mammal, said method comprising administering to said mammal an effective amount of an agent of formula (I):

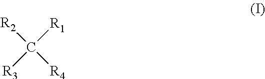

R₁ represents a saturated or unsaturated, branched or linear optionally substituted C₆-C₂₈aliphatic group;
R₂ represents a C₂-C₄ alkyl substituted from 1 to 3 times with groups independently selected from
   —(CH₂)ₘOR′ where R′ is H or C₁₋₅ alkyl, —(CH₂)ₘCOOR″ where R″ is H or C₁₋₅ alkyl, —(CH₂)ₘNH₂, —(CH₂)ₘNH₃, —(CH₂)ₘN(C₁₋₆alkyl)₂, —(CH₂)ₘCHNH(COR′″)—(CH₂)ₚOH, —(CH₂)ₘN(C₁₋₆alkyl)₃, —(CH₂)ₘP(O)(OH)₂, —(CH)(—NHC(O)—C₁-C₂₈alkyl)-(CH₂)ₚ—O—P(O)(OH)—O—(CH₂)ₘ—NH₃, and —(CH)(—NHC(O)—C₁-C₂₈alkyl)-(CH₂)ₚ—O—P(O)(OH)—O—(CH₂)ₘ—N(C₁-C₄alkyl)₃;
R₃ and R₄ independently represent H or —(CH₂)ₙOR′″ where R′″ is H or C₁₋₃ alkyl;

m iso, 1, or 2;

n is 0, 1, or 2;

p is 1 or 2;

or a pharmaceutically acceptable salt thereof.

In one embodiment, said agent is a non-acylated, basic sphingolipid which is not phosphorylated.

In another embodiment R₁ represents a C₆-C₂₈ aliphatic chain derived from the following fatty acids:
saturated fatty acids: (e.g., caproic, caprylic, perlargonic, capric, lauric, myristic, palmitic, daturic, steric, arachidic, behenic, lignoceric, cerotic, carboceric and montanic acid),
mono or multi-branched-chain fatty acids: (e.g., phytomonic, laetiporic, mycoceranic, mycocerosic, phthioceranic, pristanic, and retinoic acid),
branched methoxy fatty acids: (e.g., 2-methoxy-14-methylpentadecanoic acid),
cis and trans monoenoic fatty acids: (e.g., caproleic, lauroleic, linderic, sapienic, petroselenic, oleic, elaidic, vaccenic, gadoleic, ondoic, cetoleic, erucic, nervonic, and t3-hexadecanoic acid),
ring containing fatty acids: (e.g., lactobaeillic, majusculoic, gorlic, hydnocarpic, chaulmoogric, 11-cyclohexylundecanoic, 13-cyclohexyltridecanoic, 10,13-epoxy-11-methyloctadeca-10,12-dienoic, and lipoic acid),
acetylenic and polyacetylenic fatty acids: (e.g., tariric, santalbic, 6,9-octadecenynoic, crepenynic, scleropyric, phomallenic, and oropheic acid),
polyenoic fatty acids: (e.g., linoleic, arachidonic, and nisinic acid),
hydroxyl, thio, halo, nitro, arseno, and phosphorus containing fatty acids: (e.g., 19-fluoro-oleic, 9-chloro-10-hydroxypalmitic, and 12-nitro-9-cis, 12-cis-octadecadienoate acid).

In yet another embodiment R₃ is OH and R₄ is H.

In still another embodiment both R₃ and R₄ are H.

In a further embodiment R₃ is OH and R₄ is H and R₂ is selected from:

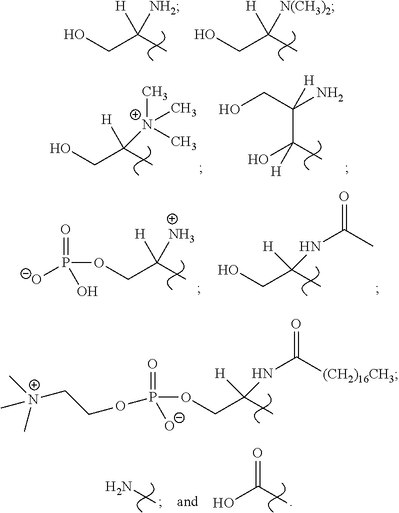

In another embodiment R₁ represents a saturated or unsaturated linear substituted C₁₂-C₂₈ alkyl, R₂ represents —(CH₂)ₘNH₃, —(CH₂)ₘN(C₁₋₆ alkyl)₃, m is 0, and R₃ and R₄ represent H. Accordingly, it will be appreciated that in an embodiment the agent may be a quartenary ammonium compound. Examples of such compounds include known cationic surfactant molecules, for example, hexadecyl-trimethyl ammonium (‘C16-TMA’) (and halide salt forms thereof such as CTAB and CTAC).

In still another embodiment, said agent is:

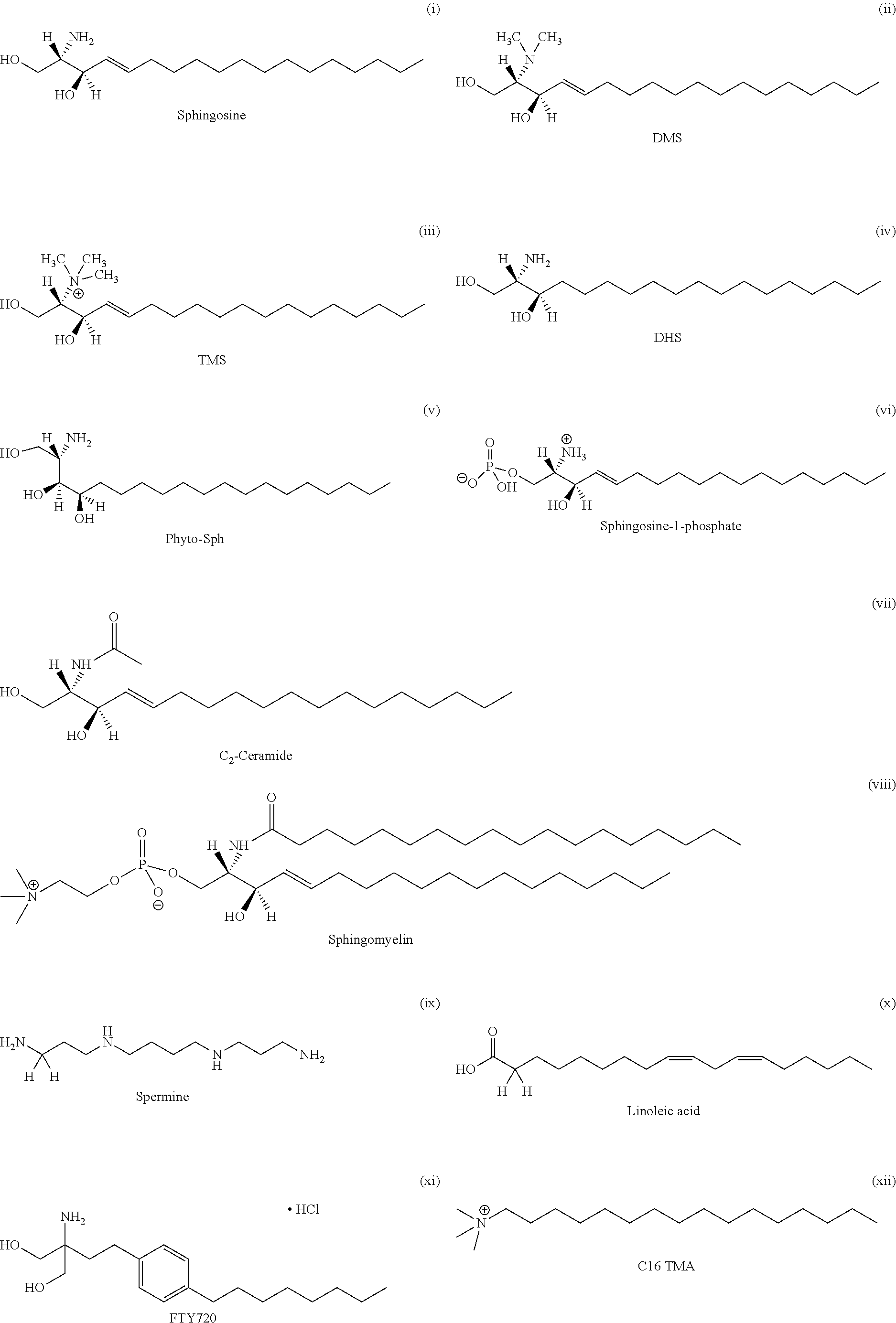

It has been still further determined by the inventors that although FTY720 can function as a sphingosine mimetic in the context of the present invention, this particular molecule in fact exhibits significant unwanted immunosuppressive side effects. To this end the findings of the present invention are particularly valuable since they now enable the design and screening for agents which exhibit the ability to effect protein 14-3-3 dimer disruption, and thereby enable Ser58 or analogous residue phosphorylation, without immunosuppressive side effects. Since the structural features required for a molecule to selectively enable phosphorylation of Ser58 or analogous residue were previously unknown, the present invention has, for the first time, provided both the necessary structural features of an agent suitable to achieve the desired functional outcome and an assay to test for this functional activity.

In one embodiment, said agent is not FTY720. In another embodiment, it is not phosphorylated FTY720. In yet another embodiment, where the condition being treated is a neoplastic condition, said agent is not FTY720. In yet another embodiment, it is not phosphorylated 720.

A further aspect of the present invention relates to the use of the invention in relation to the treatment or prophylaxis of disease conditions. Without limiting the present invention to any one theory or mode of action, the range of cellular activities which are regulated by protein 14-3-3 renders the regulation of the phosphorylation of this molecule an integral component of both healthy and disease state physiological processes. As detailed hereinbefore, protein 14-3-3 is involved, inter alfa, in the regulation of cell division, growth and apoptosis. Accordingly, the method of the present invention provides a valuable tool for modulating aberrant or otherwise unwanted cellular proliferation which is regulated via protein 14-3-3.

Accordingly, yet another aspect of the present invention is directed to a method for the treatment or prophylaxis of a condition in a mammal, which condition is characterised by inappropriate protein 14-3-3 or homologue or variant functionality said method comprising administering to said mammal an effective amount of an agent which either modulates the interaction of sphingosine or homologue or variant with said protein 14-3-3 or which mimics the sphingosine interaction wherein agonising said interaction facilitates phosphorylation of Ser58 or analogous residue thereby inhibiting the functionality of said protein 14-3-3 and wherein antagonising said interaction inhibits phosphorylation of Ser58 or analogous residue thereby maintaining said protein 14-3-3 functionality.

In yet another aspect, the present invention is directed to a method for regulating cell division, cell growth or cellular apoptosis in a mammal said method comprising administering to said mammal an effective amount of an agent which either modulates the interaction of sphingosine or homologue or variant with protein 14-3-3 or which mimics the sphingosine interaction wherein agonising said interaction facilitates phosphorylation of Ser58 or analogous residue thereby inhibiting the functionality of said protein 14-3-3 and wherein antagonising said interaction inhibits phosphorylation of Ser58 or analogous residue thereby maintaining said protein 14-3-3 functionality.

More particularly, there is provided a method for treating a condition characterised by unwanted cellular division, said method comprising administering to said mammal an effective amount of an agent which either agonises the interaction of sphingosine or homologue or variant with protein 14-3-3 or homologue or variant or mimics the sphingosine interaction wherein agonising said interaction facilitates phosphorylation of Ser58 or analogous residue thereby inducing cellular apoptosis.

In one embodiment, said condition is a neoplastic condition.

In one embodiment said serine 58 or analogous residue is selected from the list consisting essentially of:
(i) Ser58 of protein 14-3-3ζ;
(ii) Ser59 of protein 14-3-3η;
(iii) Ser59 of protein 14-3-3γ;
(iv) Ser59 of protein 14-3-3ε; and
(v) Ser60 of protein 14-3-3β.

In another embodiment said agent interacts with one or more of protein 14-3-3 residues D20, D21, K85, R18, E5, K74 or R55 or analogous residue.

In still another embodiment, said protein 14-3-3 is 14-3-3ζ and said residues are D20, D21, E89, K85, R13, E5, K74 or R55.

In still another embodiment, said residues are D20, D21, E89, K85 or R18.

In another embodiment, said protein 14-3-3 is selected from:

(i) protein 14-3-3γ and said residues are R19, D21, D22, R56, K88 or E92;

(ii) protein 14-3-3β and said residues are R20, D22, D23, R57, K87 or E91;

(iii) protein 14-3-3η and said residues are R19, D21, D22, R56, K88 or E92;

(iv) protein 14-3-3ε and said residues are R19, D21, E22, R56 or E92;

(v) protein 14-3-3τ and said residues are R18, D20, D21, R55, K85 or E89;

(vi) protein 14-3-3σ and said residues are R18, E20, D21, K87 or E91.

In yet another embodiment, said agent is non-phosphorylated.

In still another embodiment, said agent is a non-acylated, basic sphingolipid which is not phosphorylated.

In yet another embodiment, said agent is of formula (I):

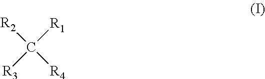

R₁ represents a saturated or unsaturated, branched or linear optionally substituted C₆-C₂₈aliphatic group;
R₂ represents a C₂-C₄ alkyl substituted from 1 to 3 times with groups independently selected from
   —(CH₂)ₘOR′ where R′ is H or C₁₋₅ alkyl, —(CH₂)ₘCOOR″ where R″ is H or C₁₋₅ alkyl, —(CH₂)ₘNH₂, —(CH₂)ₘNH₃, —(CH₂)ₘN(C₁₋₆alkyl)₂, —(CH₂)ₘCHNH(COR′″)—(CH₂)ₚOH, —(CH₂)ₘN(C₁₋₆alkyl)₃, —(CH₂)ₘP(O)(OH)₂, —(CH)(—NHC(O)—C₁-C₂₈alkyl)-(CH₂)ₚ—O—P(O)(OH)—O—(CH₂)ₘ—NH₃, and —(CH)(—NHC(O)—C₁-C₂₈alkyl)-(CH₂)ₚ—O—P(O)(OH)—O—(C H₂)ₘ—N(C₁-C₄alkyl)₃;
R₃ and R₄ independently represent H or —(CH₂)ₙOR′″ where R′″ is H or C₁₋₃ alkyl;

m is 0, 1, or 2;

n is 0, 1, or 2;

p is 1 or 2;

or a pharmaceutically acceptable salt thereof.

In another embodiment R₁ represents a C₆-C₂₈ aliphatic chain derived from the following fatty acids:
saturated fatty acids: (e.g., caproic, caprylic, perlargonic, capric, lauric, myristic, palmitic, daturic, steric, arachidic, behenic, lignoceric, cerotic, carboceric and montanic acid),
mono or multi-branched-chain fatty acids: (e.g., phytomonic, laetiporic, mycoceranic, mycocerosic, phthioceranic, pristanic, and retinoic acid),
branched methoxy fatty acids: (e.g., 2-methoxy-14-methylpentadecanoic acid),
cis and trans monoenoic fatty acids: (e.g., caproleic, lauroleic, linderic, sapienic, petroselenic, oleic, elaidic, vaccenic, gadoleic, ondoic, cetoleic, erucic, nervonic, and t3-hexadecanoic acid),
ring containing fatty acids: (e.g., lactobaeillic, majusculoic, gorlic, hydnocarpic, chaulmoogric, 11-cyclohexylundecanoic, 13-cyclohexyltridecanoic, 10,13-epoxy-11-methyloctadeca-10,12-dienoic, and lipoic acid),
acetylenic and polyacetylenic fatty acids: (e.g., tariric, santalbic, 6,9-octadecenynoic, crepenynic, scleropyric, phomallenic, and oropheic acid),
polyenoic fatty acids: (e.g., linoleic, arachidonic, and nisinic acid),
hydroxyl, thio, halo, nitro, arseno, and phosphorus containing fatty acids: (e.g., 19-fluoro-oleic, 9-chloro-10-hydroxypalmitic, and 12-nitro-9-cis, 12-cis-octadecadienoate acid).

In yet another embodiment R₃ is OH and R₄ is H.

In still another embodiment both R₃ and R₄ are H.

In a further embodiment R₃ is OH and R₄ is H and R₂ is selected from:

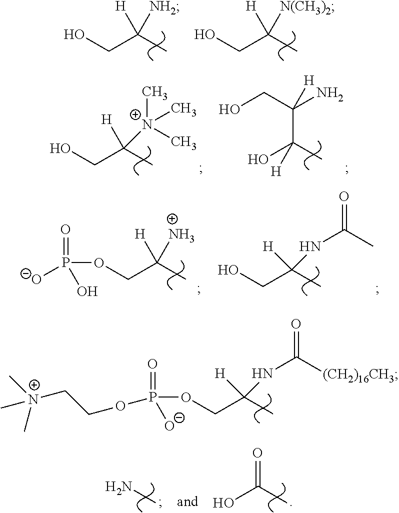

In another embodiment R₁ represents a saturated or unsaturated linear substituted C₁₂-C₂₈ alkyl, R₂ represents —(CH₂)ₘNH₃, —(CH₂)ₘN(C₁₋₆ alkyl)₃, m is 0, and R₃ and R₄ represent H. Accordingly, it will be appreciated that in an embodiment the agent may be a quartenary ammonium compound. Examples of such compounds include known cationic surfactant molecules, for example, hexadecyl-trimethyl ammonium (‘C16-TMA’) (and halide salt forms thereof such as CTAB and CTAC).

In still another embodiment, said agent is:

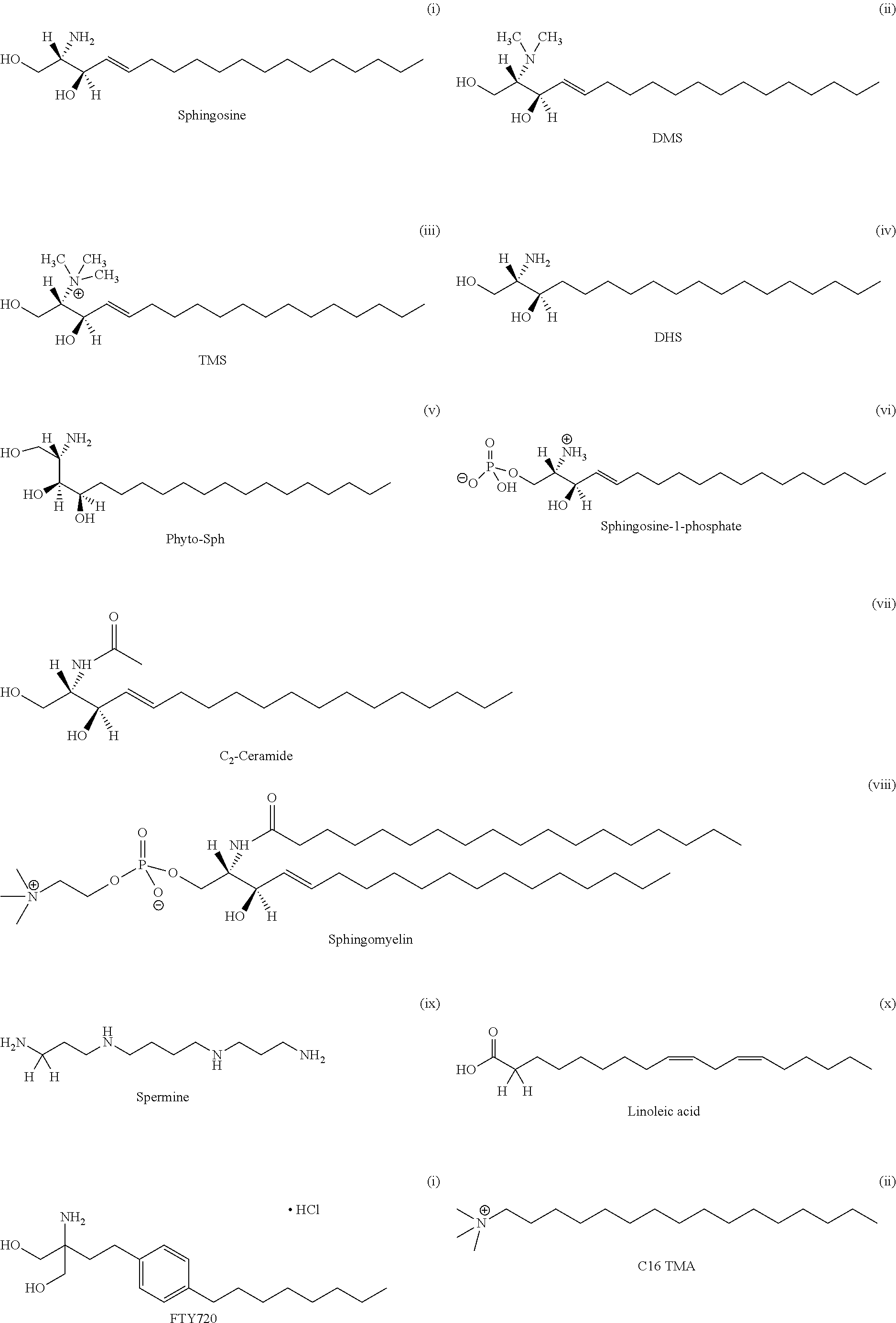

As detailed hereinbefore, the binding of sphingosine to protein 14-3-3 enables phosphorylation of protein 14-3-3, which then renders protein 14-3-3 monomeric and thereby unable to bind to and sequester pro-apoptotic molecules such as BAD, ASK-1 and FKHR. The cells are thereby not protected from apoptotic signals.

Reference to “unwanted cellular division or cell growth” should be understood as a reference to overactive cellular proliferation, insufficient cellular proliferation or to physiologically normal cellular activity which is inappropriate in that it is unwanted. For example, to the extent that a cell is neoplastic, it is desirable that the promotion of cellular proliferation and anti-apoptotic characteristics be downregulated. Neoplastic conditions which can be treated in accordance with the method of the present invention include those of the nervous system tumours, retinoblastoma, neuroblastoma and other paediatric tumours, head and neck cancers (eg. squamous cell cancers), breast and prostate cancers, lung cancer (both small and non-small cell lung cancer), kidney cancers (eg. renal cell adenocarcinoma), brain cancer, lung cancer, stomach cancer, oesophagogastric cancers, hepatocellular carcinoma, pancreaticobiliary neoplasias (eg. adenocarcinomas and islet cell tumours), colorectal cancer, cervical, and anal cancers, uterine and other reproductive tract cancers, urinary tract cancers (eg. of ureter and bladder), germ cell tumours (eg. testicular germ cell tumours or ovarian germ cell tumours), ovarian cancer (eg. ovarian epithelial cancers), carcinomas of unknown primary, human immunodeficiency associated malignancies (eg. Kaposi's sarcoma), lymphomas, leukemias, malignant melanomas, sarcomas, endocrine tumours (eg. of thyroid gland), mesothelioma and other pleural tumours, neuroendocrine tumours and carcinoid tumours. Similarly, diseases which are characterised by inflammation, such as rheumatoid arthritis, atherosclerosis, asthma, autoimmune disease and inflammatory bowel disease, are known to involve cellular activation by cytokines such as TNF, leading to the synthesis and secretion of inflammatory mediators, such as adhesion molecules. In such situations, it is also desirable to downregulate cell functionality, such as by inducing apoptosis of these cells.

Although one embodiment is to downregulate cellular growth or division, it should be understood that one may also seek to upregulate said cellular activity, for example to promote angiogenesis or to prevent neurodegeneration or other unwanted tissue death, such as cardiac tissue death. For example, neurodegenerative conditions, such as Parkinson's disease, Alzheimer's disease, Creutzfeldt-Jakob disease and transmissible spongiform encephalopathies may be characterised by an element of unwanted neuronal cell death. Similarly, heart disease is characterised by unwanted cardiac tissue death, such as in the context of myocardial infarction, angina, diabetic cardiomyopathy, hibernating myocardium in chronic ischaemia (e.g. post-stroke condition). In this situation, an antagonist of the interaction of sphingosine/protein 14-3-3 would be sought to be used.

Accordingly, in another embodiment there is provided a method for treating a condition characterised by unwanted cell death, said method comprising administering to said mammal an agent which antagonises the interaction of sphingosine or homologue or variant with protein 14-3-3-or homologue or variant wherein antagonising said interaction inhibits phosphorylation of Ser58 or analogous residue thereby inhibiting cellular apoptosis.

In one embodiment said serine 58 or analogous residue is selected from the list consisting essentially of:
(i) Ser58 of protein 14-3-3ζ;
(ii) Ser59 of protein 14-3-3η;
(iii) Ser59 of protein 14-3-3γ;
(iv) Ser59 of protein 14-3-3ε; and
(v) Ser60 of protein l4-3-3β.

In another embodiment said antagonist is an antibody, siRNA, antisense RNA, miRNA or aptamer directed to either protein 14-3-3 or sphingosine. In yet another embodiment, said antagonist is a ribozyme or DNAzyme.

The term “mammal” as used herein includes humans, primates, livestock animals (eg. sheep, pigs, cattle, horses, donkeys), laboratory test animals (eg. mice, rabbits, rats, guinea pigs), companion animals (eg. dogs, cats) and captive wild animals (eg. foxes, kangaroos, deer). Preferably, the mammal is human or a laboratory test animal. Even more preferably, the mammal is a human.

An “effective amount” means an amount necessary at least partly to attain the desired response, or to delay the onset or inhibit progression or halt altogether, the onset or progression of a particular condition being treated. The amount varies depending upon the health and physical condition of the individual to be treated, the taxonomic group of individual to be treated, the degree of protection desired, the formulation of the composition, the assessment of the medical situation, and other relevant factors. It is expected that the amount will fall in a relatively broad range that can be determined through routine trials.

Reference herein to “treatment” and “prophylaxis” is to be considered in its broadest context. The term “treatment” does not necessarily imply that a subject is treated until total recovery. Similarly, “prophylaxis” does not necessarily mean that the subject will not eventually contract a disease condition. Accordingly, treatment and prophylaxis include amelioration of the symptoms of a particular condition or preventing or otherwise reducing the risk of developing a particular condition. The term “prophylaxis” may be considered as reducing the severity or onset of a particular condition. “Treatment” may also reduce the severity of an existing condition.

The present invention further contemplates a combination of therapies, such as the administration of the agent together with subjection of the mammal to other agents, drugs or treatments which may be useful in relation to the treatment of the subject condition such as cytotoxic agents or radiotherapy in the treatment of cancer.

Administration of the modulatory agent, in the form of a pharmaceutical composition, may be performed by any convenient means. The modulatory agent of the pharmaceutical composition is contemplated to exhibit therapeutic activity when administered in an amount which depends on the particular case. The variation depends, for example, on the human or animal and the modulatory agent chosen. A broad range of doses may be applicable. Considering a patient, for example, from about 0.1 μg to about 1 μg of modulatory agent may be administered per kilogram of body weight per day. Dosage regimes may be adjusted to provide the optimum therapeutic response. For example, several divided doses may be administered daily, weekly, monthly or other suitable time intervals or the dose may be proportionally reduced as indicated by the exigencies of the situation.

The modulatory agent may be administered in a convenient manner such as by the oral, intravenous (where water soluble), intraperitoneal, intramuscular, subcutaneous, intradermal or suppository routes or implanting (e.g. using slow release molecules). The modulatory agent may be administered in the form of pharmaceutically acceptable nontoxic salts, such as acid addition salts or metal complexes, e.g. with zinc, iron or the like (which are considered as salts for purposes of this application). Illustrative of such acid addition salts are hydrochloride, hydrobromide, sulphate, phosphate, maleate, acetate, citrate, benzoate, succinate, malate, ascorbate, tartrate and the like. If the active ingredient is to be administered in tablet form, the tablet may contain a binder such as tragacanth, corn starch or gelatin; a disintegrating agent, such as alginic acid; and a lubricant, such as magnesium stearate.

Routes of administration include, but are not limited to, respiratorally, intratracheally, nasopharyngeally, intravenously, intraperitoneally, subcutaneously, intracranially, intradermally, intramuscularly, intraoccularly, intrathecally, intracereberally, intranasally, infusion, orally, rectally, via IV drip patch and implant.

In accordance with these methods, the agent defined in accordance with the present invention may be coadministered with one or more other compounds or molecules. By “coadministered” is meant simultaneous administration in the same formulation or in two different formulations via the same or different routes or sequential administration by the same or different routes. For example, the subject agent may be administered together with an agonistic agent in order to enhance its effects. By “sequential” administration is meant a time difference of from seconds, minutes, hours or days between the administration of the two types of molecules. These molecules may be administered in any order.

In still another aspect, the present invention is directed to an agent for use in modulating protein 14-3-3 or homologue or variant functioning wherein agonising or mimicking the interaction of sphingosine with protein 14-3-3 facilitates phosphorylation of Ser58 or analogous residue thereby inhibiting the functionality of said protein 14-3-3 and wherein antagonising said interaction inhibits phosphorylation of Ser58 or analogous residue thereby maintaining said protein 14-3-3 functionality.

In yet another aspect, the present invention is directed to an agent for use in regulating cell division, cell growth, cell death or cellular apoptosis in a mammal, which agent either modulates the interaction of sphingosine or homologue or variant with protein 14-3-3 or mimics the sphingosine interaction wherein agonising said interaction facilitates phosphorylation of Ser58 or analogous residue thereby inhibiting the functionality of said protein 14-3-3 and wherein antagonising said interaction inhibits phosphorylation of Ser58 or analogous residue thereby maintaining said protein 14-3-3 functionality.

In still another aspect there is provided the use of an agent in the manufacture of a medicament for treating a condition characterised by unwanted cellular division, which agent agonises the interaction of sphingosine or homologue or variant with protein 14-3-3 or homologue or variant or mimics the sphingosine interaction wherein agonising said interaction facilitates phosphorylation of Ser58 or analogous residue thereby inducing apoptosis.

In one embodiment, said condition is characterised by unwanted cell growth or division, such as a neoplastic condition, and said agent is a sphingosine mimetic.

In one embodiment said serine 58 or analogous residue is selected from the list consisting essentially of:
(i) Ser58 of protein 14-3-3ζ;
(ii) Ser59 of protein 14-3-3η;
(iii) Ser59 of protein 14-3-3γ;
(iv) Ser59 of protein 14-3-3ε; and
(v) Ser60 of protein 14-3-3β.

In another embodiment said agent interacts with one or more of protein 14-3-3 residues D20, D21, K85, R18, E5, K74, E89 or R55 or analogous residue.

In still another embodiment, said protein 14-3-3 is 14-3-3ζ and said residues are D20, D21, E89, K85, R18, E5 or K74.

In still another embodiment, said residues are D20, D21, E89, K85 or R18.

In another embodiment, said protein 14-3-3 is selected from:

(i) protein 14-3-3γ and said residues are R19, D21, D22, R56, K88 or E92;

(ii) protein 14-3-3β and said residues are R20, D22, D23, R57, K87 or E91;

(iii) protein 14-3-3η and said residues are R19, D21, D22, R56, K88 or E92;

(iv) protein 14-3-3ε and said residues are R19, D21, E22, R56 or E92;

(v) protein 14-3-3τ and said residues are R18, D20, D21, R55, K85 or E89;

(vi) protein 14-3-3σ and said residues are R18, E20, D21, K87 or E91.

In yet another embodiment, said agent is non-phosphorylated.

In still another embodiment, said agonist is a non-acylated, basic sphingolipid which is not phosphorylated.

In a further embodiment, said agent is of formula (I):

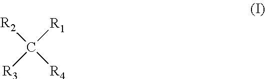

R₁ represents a saturated or unsaturated, branched or linear optionally substituted C₆-C₂₈aliphatic group;
R₂ represents a C₂-C₄ alkyl substituted from 1 to 3 times with groups independently selected from
   —(CH₂)ₘOR′ where R′ is H or C₁₋₅ alkyl, —(CH₂)ₘCOOR″ where R″ is H or C₁₋₅ alkyl, —(CH₂)ₘNH₂, —(CH₂)ₘNH₃, —(CH₂)ₘN(C₁₋₆alkyl)₂, —(CH₂)ₘCHNH(COR″′)—(CH₂)ₚOH, —(CH₂)ₘN(C₁₋₆alkyl)₃, —(CH₂)ₘP(O)(OH)₂, —(CH)(—NHC(O)—C₁-C₂₈alkyl)-(CH₂)ₚ—O—P(O)(OH)—O—(CH₂)ₘ—NH₃, and —(CH)(—NHC(O)—C₁-C₂₈alkyl)-(CH₂)ₚ—O—P(O)(OH)—O—(CH₂)ₘ—N(C₁-C₄alkyl)₃;
R₃ and R₄ independently represent H or —(CH₂)ₙOR′″ where R′″ is H or C₁₋₃ alkyl;

m is 0, 1, or 2;

n is0, 1, or 2;

p is 1 or 2;

or a pharmaceutically acceptable salt thereof.

In another embodiment R₁ represents a C₆-C₂₈ aliphatic chain derived from the following fatty acids:
saturated fatty acids: (e.g., caproic, capryiic, perlargonic, capric, lauric, myristic, palmitic, daturic, steric, arachidic, behenic, lignoceric, cerotic, carboceric and montanic acid),
mono or multi-branched-chain fatty acids: (e.g., phytomonic, laetiporic, mycoceranic, mycocerosic, phthioceranic, pristanic, and retinoic acid),
branched methoxy fatty acids: (e.g., 2-methoxy-14-methylpentadecanoic acid),
cis and trans monoenoic fatty acids: (e.g., caproleic, lauroleic, linderic, sapienic, petroselenic, oleic, elaidic, vaccenic, gadoleic, ondoic, cetoleic, erucic, nervonic, and t3-hexadecanoic acid),
ring containing fatty acids: (e.g., lactobaeillic, majusculoic, gorlic, hydnocarpic, chaulmoogric, 11-cyclohexylundecanoic, 13-cyclohexyltridecanoic, 10,13-epoxy-11-methyloctadeca-10,12-dienoic, and lipoic acid),
acetylenic and polyacetylenic fatty acids: (e.g., tariric, santalbic, 6,9-octadecenynoic, crepenynic, scleropyric, phomallenic, and oropheic acid),
polyenoic fatty acids: (e.g., linoleic, arachidonic, and nisinic acid),
hydroxyl, thio, halo, nitro, arseno, and phosphorus containing fatty acids: (e.g., 19-fluoro-oleic, 9-chloro-10-hydroxypalmitic, and 12-nitro-9-cis, 12-cis-octadecadienoate acid).

In yet another embodiment R₃ is OH and R₄ is H.

In still another embodiment both R₃ and R₄ are H.

In a further embodiment R₃ is OH and R₄ is H and R₂ is selected from:

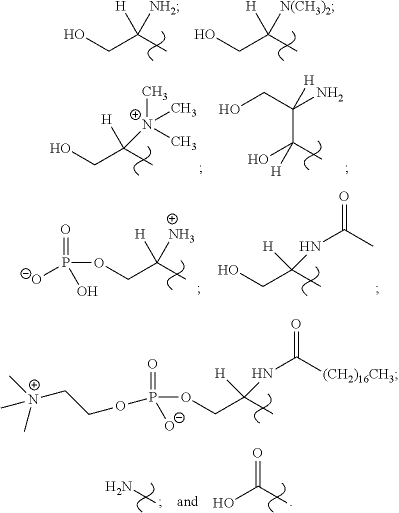

In another embodiment R₁ represents a saturated or unsaturated linear substituted C₁₂-C₂₈ alkyl, R₂ represents —(CH₂)ₘNH₃, —(CH₂)ₘN(C₁₋₆ alkyl)₃, m is 0, and R₃ and R₄ represent H. Accordingly, it will be appreciated that in an embodiment the agent may be a quartenary ammonium compound. Examples of such compounds include known cationic surfactant molecules, for example, hexadecyl-trimethyl ammonium (‘C16-TMA’) (and halide salt forms thereof such as CTAB and CTAC).

In still another embodiment, said agent is:

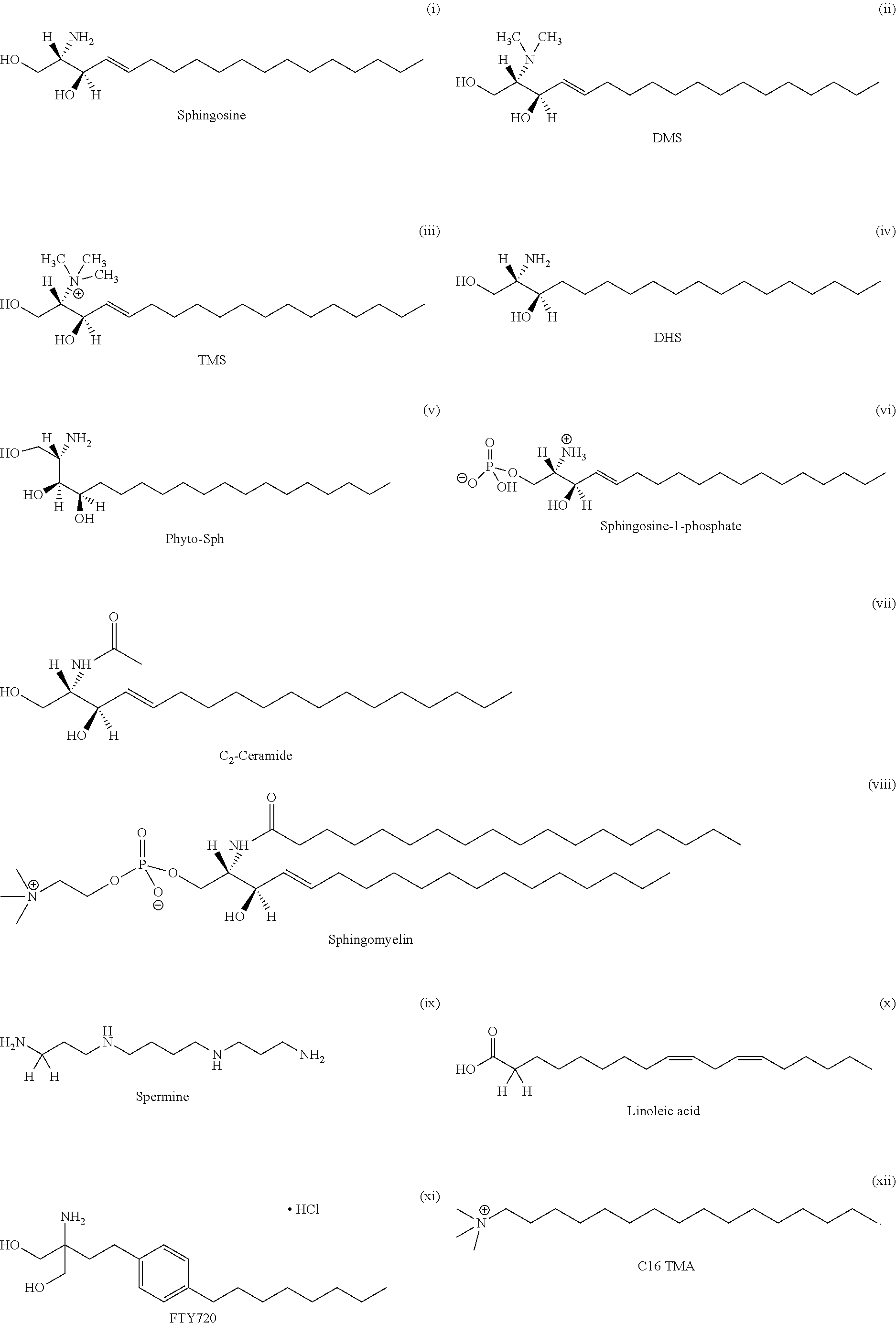

In yet still another aspect there is provided the use of an agent in the manufacture of a medicament for treating a condition characterised by unwanted cell death, which agent antagonises the interaction of sphingosine with protein 14-3-3 or homologue or variant wherein antagonising said interaction inhibits phosphorylation of Ser58 or analogous residue thereby inhibiting apoptosis.

In one embodiment said serine 58 or analogous residue is selected from the list consisting essentially of:
(i) Ser58 of protein 14-3-3ζ;
(ii) Ser59 of protein 14-3-3η;
(iii) Ser59 of protein 14-3-3γ;
(iv) Ser59 of protein 14-3-3ε; and
(v) Ser60 of protein 14-3-3β.

In yet another further aspect, the present invention contemplates a pharmaceutical composition comprising the modulatory agent as hereinbefore defined together with one or more pharmaceutically acceptable carriers and/or diluents. These agents are referred to as the active ingredients.

The pharmaceutical forms suitable for injectable use include sterile aqueous solutions (where water soluble) or dispersions and sterile powders for the extemporaneous preparation of sterile injectable solutions or dispersion or may be in the form of a cream or other form suitable for topical application. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol and liquid polyethylene glycol, and the like), suitable mixtures thereof, and vegetable oils. The proper fluidity can be maintained, for example, by the use of a coating such as lecithin, by the maintenance of the required particle size in the case of dispersion and by the use of superfactants. The preventions of the action of microorganisms can be brought about by various antibacterial and antifungal agents, for example, parabens, chlorobutanol, phenol, sorbic acid, thimerosal and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions are prepared by incorporating the active compounds in the required amount in the appropriate solvent with various of the other ingredients enumerated above, as required, followed by filtered sterilisation. Generally, dispersions are prepared by incorporating the various sterilised active ingredient into a sterile vehicle which contains the basic dispersion medium and the required other ingredients from those enumerated above. In the case of sterile powders for the preparation of sterile injectable solutions, the preferred methods of preparation are vacuum drying and the freeze-drying technique which yield a powder of the active ingredient plus any additional desired ingredient from previously sterile-filtered solution thereof.

When the active ingredients are suitably protected they may be orally administered, for example, with an inert diluent or with an assimilable edible carrier, or it may be enclosed in hard or soft shell gelatin capsule, or it may be compressed into tablets, or it may be incorporated directly with the food of the diet. For oral therapeutic administration, the active compound may be incorporated with excipients and used in the form of ingestible tablets, buccal tablets, troches, capsules, elixirs, suspensions, syrups, wafers, and the like. Such compositions and preparations should contain at least 1% by weight of active compound. The percentage of the compositions and preparations may, of course, be varied and may conveniently be between about 5 to about 80% of the weight of the unit. The amount of active compound in such therapeutically useful compositions in such that a suitable dosage will be obtained. Preferred compositions or preparations according to the present invention are prepared so that an oral dosage unit form contains between about 0.1 μg and 2000 mg of active compound.

The tablets, troches, pills, capsules and the like may also contain the components as listed hereafter: a binder such as gum, acacia, corn starch or gelatin; excipients such as dicalcium phosphate; a disintegrating agent such as corn starch, potato starch, alginic acid and the like; a lubricant such as magnesium stearate; and a sweetening agent such as sucrose, lactose or saccharin may be added or a flavouring agent such as peppermint, oil of wintergreen, or cherry flavouring. When the dosage unit form is a capsule, it may contain, in addition to materials of the above type, a liquid carrier. Various other materials may be present as coatings or to otherwise modify the physical form of the dosage unit. For instance, tablets, pills, or capsules may be coated with shellac, sugar or both. A syrup or elixir may contain the active compound, sucrose as a sweetening agent, methyl and propylparabens as preservatives, a dye and flavouring such as cherry or orange flavour. Of course, any material used in preparing any dosage unit form should be pharmaceutically pure and substantially non-toxic in the amounts employed. In addition, the active compound(s) may be incorporated into sustained-release preparations and formulations.

The pharmaceutical composition may also comprise genetic molecules such as a vector capable of transfecting target cells where the vector carries a nucleic acid molecule encoding a modulatory agent. The vector may, for example, be a viral vector.

Yet another aspect of the present invention relates to the agent as hereinbefore defined or as identified in accordance with the screening method hereinbefore described, when used in the method of the present invention.

The present invention is further described by reference to the following non-limiting examples.

### EXAMPLE 1

### Materials and Methods

### Reagents

Sphingolipids (purchased from Biomol, Pa.) and other lipids (purchased from Sigma, Mo.) utilised in these studies were routinely prepared as ethanol stocks and stored at −20° C. Prior to use, the lipids were sonicated and diluted in ethanol (final concentration (0.1% v/v). [³ H]-D-erythro-sphingosine was from Perkin Elmer. PKA catalytic subunit purified from bovine heart was purchased from Sigma. Anti-14-3-3 antibody, recombinant wild-type and mutant 14-3-3ζ were generated as described previously (Woodcock et al. 2003, J. Biol. Chem. 278:36323-27). Anti-PKC antibody was from Santa Cruz, anti-HA and anti-Flag antibodies were from Sigma. Horseradish peroxidase-conjugated secondary antibodies were purchased from Pierce. Anti-CD95 antibody CH-11 was purchased from Millipore.

### 14-3-3 Phosphorylation by PKA

Substrate 14-3-3 (either 0.5 μg of purified recombinant 14-3-3, a 10 μL aliquot of fractions from gel filtration or immunoprecipitates from 14-3-3-Myc transfected COS cells) were added to 30 μL reaction mixture comprising 0.2 Units of the PKA catalytic subunit, in the presences or absence of sphingolipid (delivered in 0.1% v/v ethanol) in PKA reaction buffer (20 mM MOPS, pH 7.4, 20 mM β-glycerol phosphate, 4 mM EGTA, 1 mM Na₃VO₄, 60 mM MgCl₂, 1 mM DTT, 500 μM ATP, 2.4 μCi [³²P]γ-ATP). Reactions were incubated at 37° C. for 15 minutes. After incubation, reactions were separated on 12.5% SDS-PAGE, coomassie stained and 14-3-3 phosphorylation analysed by autoradiography.

### Gel Filtration

Recombinant 14-3-3ζ (10 μg) was pre-incubated with 25 μM DMA delivered in 0.1% v/v ethanol and [³H]-sphingosine, (0.5 μCi) and then chromatographed on a SMART system with a Superdex 200PC 3.2/30 column (Amersham Biosciences, Australia). The system was operated at 40 μl/min using 20 mM Tris-HCl pH 7.5, 150 mM NaCl, 0.1 mM DTT, 10 mM β-mercaptoethanol as running buffer. Fractions (40 μl) were collected for 60 minutes after sample injection. Protein molecular weight standards; myoglobin, Mᵣ 17,000; ovalbumin, Mᵣ 44,000; γ-globulin, Mᵣ 158,000; and thyroglobulin, Mᵣ 670,000 were fractionated immediately prior to 14-3-3 fractionation.

### Expression Plasmid Construction and Cell Lines

An expression construct encoding the catalytic domain of PKGδ corresponding to the caspase-cleaved form of the enzyme was generated from pFLAG-PKCδ-Cat, (kindly provided by Dr Mitchell Denning, Cardinal Bemardin Cancer Center, Loyola University Medical Center, Illinois) (Denning et al. 2002, Cell Death & Diff. 9:40-52). This clone pFLAG-PKCδ-Cat, encompasses the cDNA for the entire human PKCδ catalytic domain but lacks seven amino acids at the N-terminus relative to the caspase-cleaved form (Sitailo et al. 2004, J. Invest. Dermatol. 123:434-43). A fragment was generated from this construct by PCR that recreated exactly the N-terminus of the caspase-3 cleaved protein (with an additional methionine residue for translation initiation) and was cloned into an engineered form of pRcCMV (lnvitrogen) plasmid which additionally fused the HA-epitope to the C-terminus of the protein. This construct was designated pRcCMV-PKCδCat-HA.

An expression construct for human 14-3-3ζ was generated by cloning the entire cDNA into an engineered form of pRcCMV (Invitrogen) plasmid which additionally fused the Myc-epitope to the C-terminus of the protein. This construct was designated pRcCMV-14-3-3ζ-Myc.

Jurkat cells cultured in RPMI containing 10% FBS were transfected with a pcDNA3 construct encoding 14-3-3ζ-Myc-IRES-GFP or an empty IRES-GFP vector using Lipofectamine 2000 (Invitrogen) according to the manufacturer's instructions. Cells were sorted at 24 hours post-transfection for GFP expression and then subjected to neomycin selection (using 1 mg/ml neomycin) and repeatedly sorting for GFP expression to generate stable 14-3-3-Myc expressing and vector cell lines.

A HEK293 cell line with doxycycline inducible expression of sphingosine kinase I (SK 1) was generated as previously described (Pham et al. 2008, Biotechniques in press).

COS Cell culture and Transfection

COS cells were cultured in RPMI medium containing 10% calf serum at 37° C. with 5% CO₂. Cells were seeded in 10 cm dishes the day before transfection and expression constructs pRcCMV-PKCδCat-HA, pRcCMV-14-3-3ζ-Myc or pRcCMV (mock) were transfected using Lipofectamine 2000 (Invitrogen) following the manufacturer's instructions. Using this protocol 30-50% transfection was routinely achieved as gauged by green-fluorescent protein expression. The day after transfection cells were harvested by scraping, washed in PBS and S200 cytosolic extracts were prepared as described previously (Megidish et al. 1998, J. Biol. Chem. 273:21834-45) or cells were lysed in NP40 lysis buffer (25 mM Tris-HCl, pH 7.5, 150 mM NaCl, 1 mM EDTA, 1 mM EGTA, 5 mM MgCl₂, 1 mM DTT, 1% Nonidet P-40, 10% glycerol) for immunoprecipitation.

### PKCδCat-HA Immunoprecipitation and Immunoblotting

PKCδCat-HA was immunoprecipitated from lml of transfected COS S100 cytosolic fraction after overnight incubation at 4° C. with 2 μg anti-HA antibody using 100 μl of a 50% slurry of protein A-sepharose beads for 1 hour at 4° C. Immunoprecipitates were washed 3 times in NP40 lysis buffer and then washed 3 times in PKC assay buffer (20 mM MOPS pH 7.2, 25 mM β-glycerol phosphate, 1 mM Na₃VO₄, 1 mM DTT, 1 mM CaCl₂). Expression of transfected PKCδCat-HA was confirmed following separation on 10% SDS-PAGE by western transfer and immunoblotting with anti-PKCδ (Santa Cruz). Immunoblotting was carried out as described previously (Woodcock et al. 2003, supra) and developed by enhanced chemiluminescence using proprietary reagents (Amersham Biosciences, Australia).

### PKC Activity Assays and 14-3-3 Phosphorylation

Equal amounts of PKCδCat-HA immunoprecipitate were used to assay for PKC kinase activity, or to phosphorylate recombinant 14-3-3. For PKC kinase activity, PKCδCat-HA immunoprecipitates were incubated with 0.5 μM PKC Ser²⁵ substrate (Alexis Biochemicals), in PKC assay buffer containing 9 mM MgCl₂, 60 μM ATP, 0.6 mM EGTA and 2.5 μCi [³²P] γ-ATP in the presence or absence of 50 μM DMS (delivered in 0.1% v/v ethanol) or 10 μM GF109203X (Biomol) in a total reaction volume of 40 μl at 37° C. for 10 minutes. Reactions were spotted onto phosphocellulose filters (Whatman P81), washed 3 times in 0.75% phosphoric acid and once with acetone before liquid scintillation counting. PKCδCat-HA immunoprecipitates were also assayed for the ability to phosphorylate recombinant 14-3-3. Reactions were carried out as for PKC kinase activity reactions using 0.5 μg recombinant 14-3-3 (WT or S58A mutant) in place of PKC Ser²⁵ substrate. Following incubation, reactions were separated on 12.5% SDS-PAGE, coomassie stained and 14-3-3 phosphorylation analysed by autoradiography.

Sphingosine Kinase 1 Treatment of Immunoprecipitates from Transfected COS Cells

14-3-3ζ-Myc was immunoprecipitated from transfected COS cell NP40 lysates using 5 μg anti-Myc antibody and protein A-sepharose beads essentially as described above. 14-3ζ-Myc immunoprecipitates were then washed twice in 100 mM Tris-HCl pH 7.4, 10 mM MgCl₂ and then resuspended in 100 μl of sphingosine kinase (SK) assay buffer (50 mM Tris-HCl pH 7.4, 150 mM NaCl, 2 mM Na₃VO₄, 10 mM NaF, 1 mM EDTA, 10% glycerol, 0.05% Triton X-100, 10 mM β-glycerol phosphate plus protease inhibitors (Roche complete EDTA-free)) containing 1 mM ATP and 1 μg of recombinant SKI (Pitson et al. 2002, J. Biol. Chem. 277:49545-553) and incubated at 37° C. for 30 minutes. Following SK1 treatment immunoprecipitates were washed twice in kinase buffer 10 mM Tris-HCl pH 7.4, 15 mM MgCl₂) and subjected to phosphorylation by PKA catalytic subunit essentially as described elsewhere (Gu et al. 2006, FEBS Letters 580:305-310).

### Lipid Extraction and Measurement of Sphingosine

Lipids were extracted from HEK293 cells as described previously (Ma et al. 2005, J. Biol. Chem. 280:2601 1-17). Sphingosine levels in the lipid extract were determined by the sphingosine kinase method as detailed elsewhere (Ma et al. 2005, supra).

Immunoprecipitation of 14-3-3ζ-Myc and Sphingosine Determination from Jurkat Cells

Jurkat cells stably expressing either 14-3-3ζ-Myc-IRES-GFP or 1RES-GFP alone (vector) were subjected to Fas-induced apoptosis using anti-Fas antibody (anti-CD95 CH11 at 50 ng/ml) under serum-free conditions as described previously (Cuvillier et al. 2000, supra). Cells were harvested over a time-course of Fas treatment and the cell pellets subjected to lysis using NP40 lysis buffer and anti-Myc immunoprecipitation carried out essentially as described above with the exception that μMace™ anti-Myc microbeads and μ columns (Miltenyi Biotec) were employed to capture the immunoprecipitated proteins and co-associated lipids. Immunoprecipitated proteins were eluted from the magnetic columns into SK assay buffer and the associated sphingosine determined as described elsewhere (Ma et al. 2005, supra).

### Results

### The Phosphorylation of 14-3-3ζ by PKA is Sphingosine-Dependent

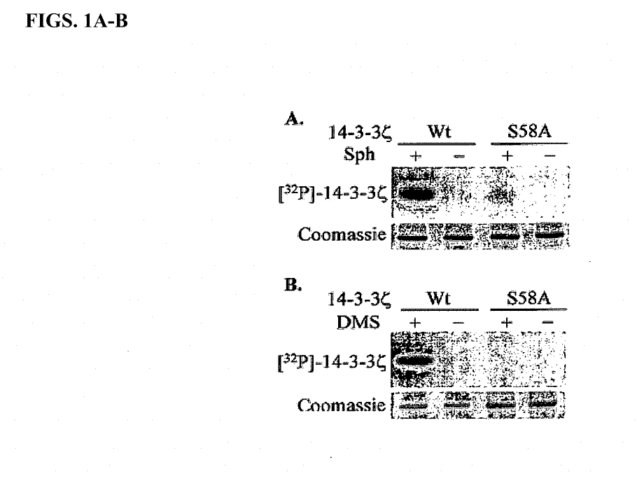

Experiments were performed to examine the ability of PKA to phosphorylate 14-3-3ζ in the absence or presence of sphingosine and its naturally occurring analogue dimethyl-sphingosine, DMS. To ascertain the role of sphingosine in 14-3-3 phosphorylation a PKA catalytic subunit was used, the active form of which is not affected by sphingosine (Ma et al. 2005, supra). FIG. 1A shows that recombinant 14-3-3ζ was not phosphorylated by PKA catalytic subunit unless sphingosine was present, demonstrating the dependency of 14-3-3 phosphorylation on sphingosine. Furthermore, alanine substitution of Ser58 abolished the sphingosine-dependent phosphorylation of 14-3-3ζ, confirming that Ser58 is the sole phosphorylation site for PKA in 14-3-3ζ. FIG. 1B shows that the sphingosine analogue dimethyl-sphingosine behaves identically to sphingosine in allowing phosphorylation of 14-3-3ζ on Ser58. These results indicate that an active kinase (i.e. PKA catalytic subunit) is insufficient for 14-3-3 phosphorylation and indicates that sphingosine is required to render 14-3-3 available for phosphorylation.

### Sphingosine and Its Analogises Render 14-3-3 Phosphorylatable by PKA Catalytic Subunit

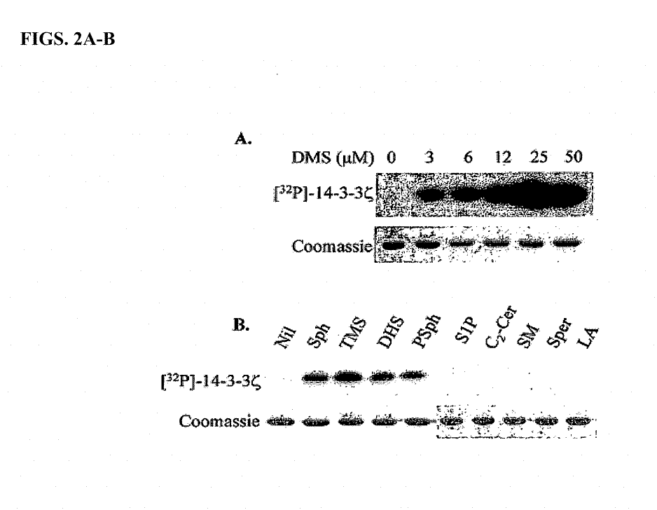

In a dose-response analysis 14-3-3ζ is increasingly phosphorylatable by PKA catalytic subunit when incubated with increasing concentrations of DMS (FIG. 2A, upper panel). This demonstrates that phosphorylation of 14-3-3ζ is sensitive to the presence of DMS with no detectable phosphorylation in the absence of lipid, robust phosphorylation seen at relatively low sphingosine concentrations (3-6 μM) and maximal phosphorylation achieved in the presence of 25 μM DMS (FIG. 2A).

To investigate the specificity of sphingosine's effect on 14-3-3, a panel of sphingolipids and related molecules were examined for their effect on 14-3-3ζ's phosphorylation by PKA catalytic subunit. FIG. 2B shows that in addition to DMS and sphingosine, trimethyl-sphingosine, dihydro-sphingosine and phyto-sphingosine, also allowed phosphorylation of 14-3-3ζ by PKA catalytic subunit. In contrast, sphingosine-1 -phosphate, ceramide, sphingomyelin, spermide, and linoleic acid did not support the phosphorylation of 14-3-3 (FIG. 2B) indicating that the substrate modulation of 14-3-3 is specific to non-acylated basic sphingolipids. The lipid modulation of 14-3-3ζ occurs only with lipids carrying a net positive charge, suggesting that basic sphingosines interact directly with the acidic 14-3-3 substrate.

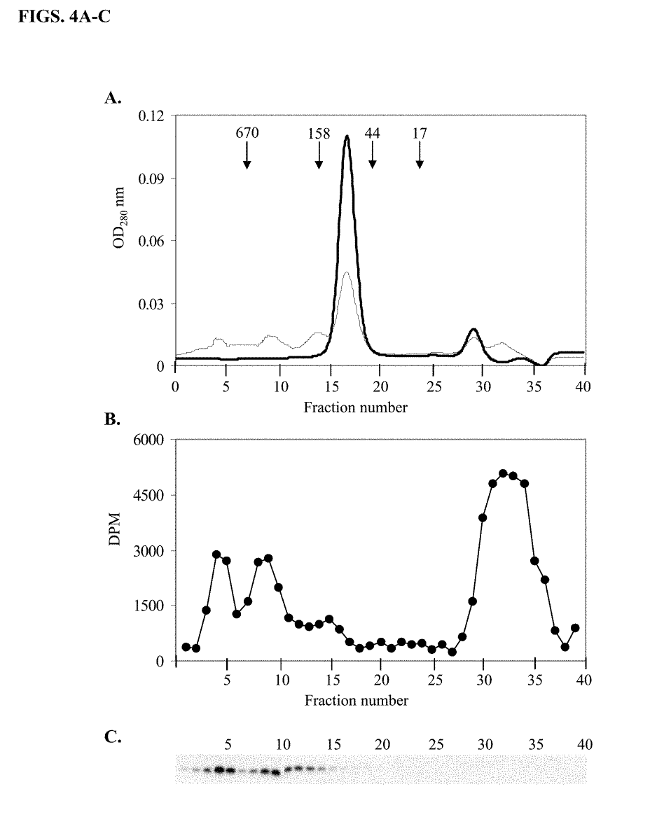

Multiple isoforms of 14-3-3 bind sphingosines and become phosphorylatable in helix three. The Ser58 phosphorylation site in 14-3-3ζ is not unique to this 14-3-3 isoform and the primary sequence surrounding this residue is highly conserved in all seven mammalian isoforms (FIG. 4A), being located within the third helix of the protein and intimately involved in dimer formation [Gardino et al., 2006, Seminars in Cancer Biology 16:173-182]. Therefore we tested in vitro whether other 14-3-3 isoforms were phosphorylatable and whether sphingosine was required for the phosphorylation. Using PKA catalytic subunit, the active form of the enzyme that is unaffected by sphingosine [Ma 2005] we found that all isoforms tested with the exception of 14-3-3σ were phosphorylatable but only in the presence of sphingosine or its analogue, dimethyl-sphingosine (DMS) (Fig.4B). I4-3-3σ does not contain a phosphorylatable residue at the 58 position (FIG. 4A), thus confirming that the Ser58 is the sole site of sphingosine-dependent PICA phosphorylation in 14-3-3.

Direct Interaction Between Sphingosine and 14-3-3ζ is Required for 14-3-3's Phosphorylation

At concentrations of 20 μM and above, sphingosine and DMS altered the behaviour of 14-3-3 on gel filtration, causing the protein to elute at molecular weight positions greater than the size of dimeric 14-3-3. This is presumably due to the formation of sphingosine micelles, consistent with the published critical micelle concentration of 18 μM (Contreras et al. 2006, Biophys. J. 90:4085-4092) and represents the interaction of 14-3-3 with sphingosine. This ability of sphingosine to accelerate the elution of 14-3-3 on gel filtration was used to separate sphingosine-bound from sphingosine-free 14-3-3 to examine the sensitivity of the two pools of 14-3-3 to phosphorylation by PKA catalytic subunit. As shown by the absorbance profile in FIG. 4A in the absence of sphingosine, 14-3-3ζ eluted as a single peak (fractions 15-18, thick solid line) at a position corresponding to a molecular weight of 60 kDa consistent with the size of dimeric 14-3-3ζ. A second minor absorbance peak (FIG. 4A, fractions 27-31) was also detected that was seen in all elution profiles and is associated with the presence of DTT in the elution buffer. Pre-incubation of 14-3-3ζ with 25 μM DMS containing a trace amount of [³H]-sphingosine resulted in a change in absorbance profile of the eluted 14-3-3ζ with a reduced dimeric 14-3-3ζ peak (FIG. 4A, fractions 15-18) and the appearance of protein absorbance peaks at positions corresponding to higher molecular weight complexes (FIG. 4A, fractions 3-14). The fractions were assayed for [³H]-sphingosine by scintillation counting. Peaks of radioactivity were detected between gel filtration fractions 3-14 and 29-36 (FIG. 4B). The elution of [³H]-sphingosine in fractions 3-14 (FIG. 4B) is very similar in profile to the elution of high molecular weight species of 14-3-3ζ (as determined by absorbance at 280 nm, FIG. 4A) and presumably corresponds to [³H]-sphingosine directly bound to 14-3-3. The second peak of radioactivity observed in gel fractions 29-36 corresponds to free [³H]-sphingosine (FIG. 4B) as a replicate gel filtration run with [³H]-sphingosine alone showed a similar radioactive peak eluting at the same position (data not shown). Very little radioactivity was detected in fractions eluted at the position of native 14-3-3 dimer (FIGS. 4A and B, fractions 15-18) indicating that all the sphingosine-bound 14-3-3 was in a multi-molecular form (FIGS. 4A and B, fractions 3-14).

In order to determine which form of 14-3-3 is phosphorylatable, the gel filtration fractions were phosphorylated in vitro by the catalytic subunit of PKA. As the data in FIG. 4C shows, only the 14-3-3 co-eluting with sphingosine (fractions 3-14), was phosphorylated by PKA catalytic subunit whereas sphingosine-free 14-3-3 (fractions 15-18) was not phosphorylatable. This indicates that the binding of sphingosine to 14-3-3 is required for phosphorylation by PKA.

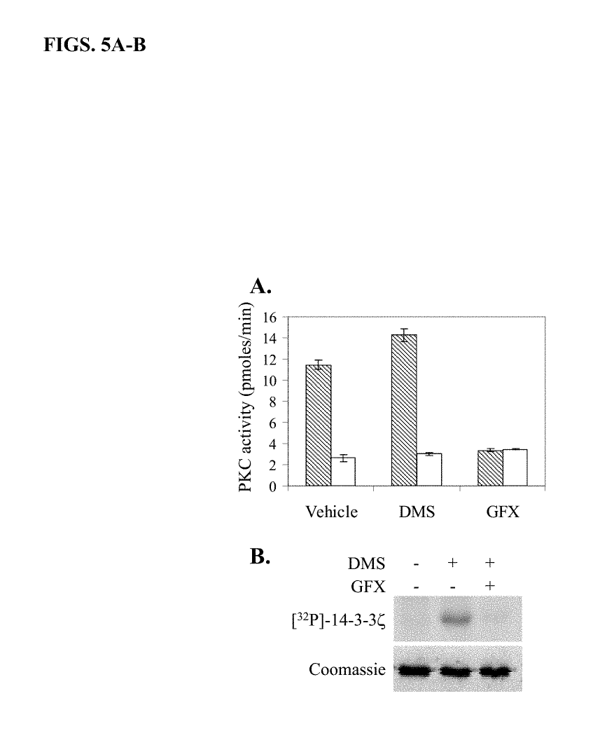

Sphingosine modulation of 14-3-3 is also required for phosphorylation by truncated PKGδ COS cells were transfected with a construct encoding an HA-tagged truncated form of PKCS (PKCδCat-HA) corresponding to the caspase-generated fragment of PKCδ (Sitailo et al. 2004, supra). The enzyme was immunoprecipitated using an anti-HA antibody and expression confirmed by immunoblotting with anti-PKCδ antibody. Kinase assays were performed with mock and PKCδCat-HA immunoprecipitates using either a PKC peptide substrate (FIG. 5A), ore recombinant 14-3-3ζ (FIG. 5B). FIG. 5A shows that immunoprecipitated PKCδCat is constitutively active as shown previously (Denning et al. 2002, supra), completely inhibited by the PKC inhibitor GF109203X, and that 50 μM DMS has no significant effect on the activity of the kinase. However, in relation to 14-3-3 phosphorylation, no phosphorylation is observed with PKCδCat unless sphingosine is present (FIG. 5B). Assays performed with the 14-3-3ζ mutant, S58A confirmed that the site of phosphorylation by PKCδ is Ser58 as suggested previously (Hamaguchi et al. 2003, supra). These results suggest that sphingosine plays a role in making 14-3-3 available for phosphorylation by caspase-cleaved PKCδ and not in activation of an already active kinase.

### Sphingosine Kinase Alters the Phosphorylatability of 14-3-3

The interaction of sphingosine with 14-3-3 determines the phosphorylatability of 14-3-3 on Ser58 in vitro. 14-3-3ζ-Myc was immunoprecipitated from transfected COS cells and phosphorylated in vitro with PKA catalytic subunit as described in the recent paper (Gu et al.

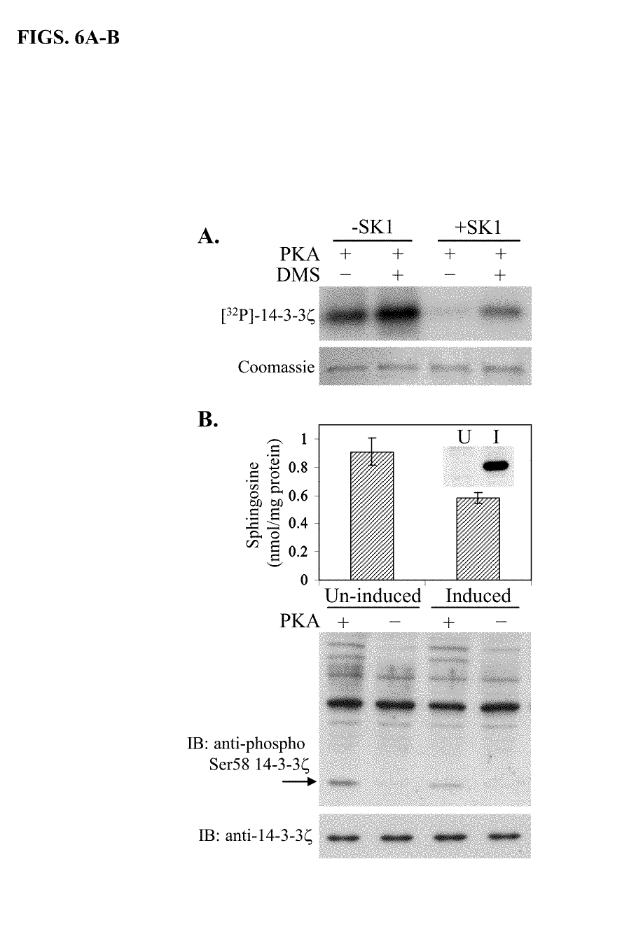

2006, supra). It was found that the immunoprecipitated 14-3-3ζ-Myc was phosphorylated in vitro by PKA catalytic subunit without addition of exogenous sphingosine (FIG. 6A, upper panel, lane 1). Additionally, the immunoprecipitated 14-3-3ζ-Myc was phosphorylated more strongly when DMS was added to the in vitro PKA reaction (FIG. 6A, upper panel, lane 2) indicating that any endogenous sphingosine is not at saturating levels.

To investigate whether the in vitro phosphorylation of immunoprecipitated 14-3-3ζ-Myc by PKA catalytic subunit is due to associated endogenous sphingosine, in vitro treatment of immunoprecipitated 14-3-3 with recombinant sphingosine kinase 1 (SK1) was carried out . Thus any 14-3-3-associated sphingosine would be converted to sphingosine-1-phosphate (Pitson et al. 2002, supra), a sphingosine-derivative that does not support 14-3-3's phosphorylation (FIG. 2B). 14-3-3ζ-Myc immunoprecipitated from transfected COS cells was incubated in vitro with recombinant SK1 and subsequently washed free of SK1 prior to in vitro PKA phosphorylation. In contrast to non-SK1 treated 14-3-3, the phosphorylation of SK1 treated immunoprecipitated 14-3-3ζ-Myc was completely abolished (FIG. 6A, upper panel, lane 3), indicating that SK1 treatment had reversed the phosphorylatability of the 14-3-3. However addition of exogenous DMS to the phosphorylation reaction after SK1 treatment restored phosphorylatability (FIG. 6A, upper panel, lane 4), emphasising the dependence of 14-3-3 phosphorylation on the sphingosine:14-3-3 interaction. Coomassie staining of the SDS-PAGE resolved in vitro phosphorylation reactions confirms that the differences in in vitro phosphorylatability of 14-3-3 are not due to differences in amount of 14-3-3ζ-Myc immunoprecipitated (FIG. 6A, lower panel).

Having demonstrated that in vitro SKI treatment could alter the phosphorylatability of 14-3-3, these studies were extended to examine the ability of SK1 to control the sphingosine:14-3-3 interaction in vivo. A doxycycline-inducible system was used to induce SKI expression in HEK293 cells. Cells were analysed in the un-induced and induced state for levels of sphingosine and expression of SK1. FIG. 6B (upper panel) shows that in the presence (induced) compared with absence of doxycycline (un-induced) sphingosine levels were reduced by 30%. This reduction in sphingosine coincides with the expression of SK1 as shown by western blotting (FIG. 6B, upper panel, inset). Lysates from the un-induced and induced cells were analysed for phosphorylatable 14-3-3 by in vitro PKA phosphorylation and immunoblotting with a phospho-specific antibody raised to Ser 58 of 14-3-3ζ (FIG. 6B). Strong 15 Ser58 phosphorylation was detected in the un-induced lysates when incubated with PKA (FIG. 6B, middle panel, lane 1) whereas in the absence of PKA only minimal phospho-Ser58 14-3-3 could be detected (FIG. 6B, middle panel, lane 2) indicating that the signal detected with PKA treatment represents 14-3-3 phosphorylated in vitro and not 14-3-3 previously phosphorylated in vivo. The level of Ser58 phosphorylation achieved with in vitro PKA treatment of lysates from induced cells was reduced relative to the un-induced sample (FIG. 6B, middle panel, compare lane 3 with lane 1). Immunoblotting of the in vitro phosphorylation reactions with an anti-14-3-3 antibody demonstrates that the change in phosphorylatability is not due to the amount of 14-3-3 protein present (FIG. 6B, lower panel). Hence the reduction in 14-3-3 phosphorylatability (FIG. 6B, middle panel) is consistent with the reduction in sphingosine level observed (FIG. 6B, upper panel) supporting the notion that SK1 controls the level of sphingosine available for interaction with 14-3-3 in vivo.

### Sphingosine Binding to 14-3-3 Fluctuates Under Physiological Conditions

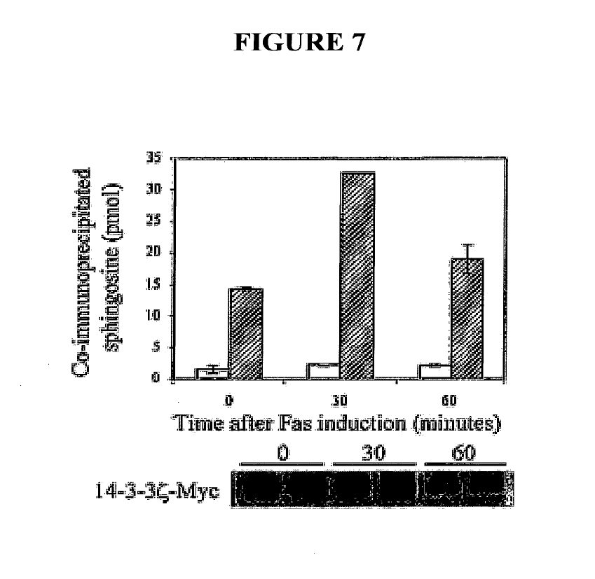

The results demonstrate the interaction of sphingosine with 14-3-3 in cells, indicating that 14-3-3 may be sensitive to changes in sphingosine levels under physiological conditions. Sphingosine levels associated with immunoprecipitated 14-3-3 were determined by quantitative conversion of S-1-P (Ma et al. 2005, supra). As shown in FIG. 7 detectable levels of sphingosine were associated with material immunoprecipitated from the 14-3-3ζ-Myc expressing cells, indicating that sphingosine is associated with immunoprecipitated 14-3-3. Furthermore, 30 minutes after induction of Fas signalling the sphingosine associated with immunoprecipitated 14-3-3 increased 2 fold and 60 minutes after induction the sphingosine level returned to unstimulated levels.

### Sphingosine-Dependent Phosphorylation Mediates Apoptosis Induced by DMS and FTY720

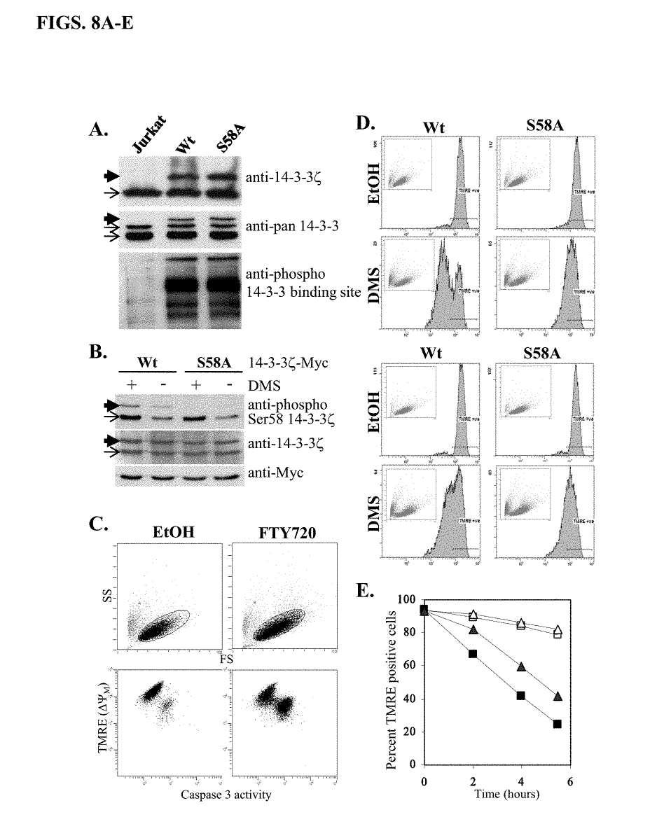

To assess the functional significance of sphingosine-dependent 14-3-3 phosphorylation we utilised Jurkat cells that undergo mitochondrial-mediated apoptosis in response to sphingosine, DMS (Cuvillier et al., J. Biol. Chem. 275 (2000), p. 15691) and FTY720 (Nagahara et al. Br. J. Pharmcol. 137 (2002), p. 953). Jurkat cells expressing either wild type or non-phosphorylatable S58A 14-3-3ζ-Myc-IRES-GFP were generated by lentiviral gene transfer. This was necessary as endogenous levels of 14-3-3 proteins are high (representing up to 0.1% of cellular protein) and therefore exogenous 14-3-3 needed to be expressed at significant levels in order to manifest any functional differences. Cells were sorted for GFP expression and immunoblotting showed that Myc-tagged 14-3-3ζ protein (FIG. 8A, upper panel, as indicated by the solid arrow head) was expressed at a comparable level to endogenous 14-3-3ζ (FIG. 8A, upper panel, as indicated by the open arrow head). Immunoblotting 14-3-3ζ-Myc wild type and S58A immunoprecipitates with an antibody that detects proteins bearing 14-3-3-binding phospho-motif confirmed that the pattern of proteins associated with either 14-3-3ζ wild type or S58A was ostensibly identical (FIG. 8A, lower panel), indicating that the phospho-serine protein binding function of 14-3-3 is unaffected by the substitution of Ser58 with Ala. In vitro phosphorylation of immunoprecipitated 14-3-3ζ-Myc by PKA catalytic subunit confirmed that wild type 14-3-3ζ-Myc and endogenous 14-3-3 isoforms are phosphorylatable either in the presence or absence of DMS but as expected S58A 14-3-3ζ-Myc protein was not (FIG. 8B).

Having confirmed appropriate expression and behaviour of Myc-tagged wild type and S58A 14-3-3ζ in the Jurkat lines, these cells were utilised in functional studies to determine the effect of S58A 14-3-3ζ expression on cell survival in response to sphingosine analogues. We utilised TMRE staining to monitor the mitochondrial permeability transition (ΔΨ_{M}) associated with apoptosis. Using this approach we observed that Jurkat cells underwent rapid commitment to apoptosis as detected by the loss in TMRE staining by 4 h of stimulation with either 2 μM DMS or FTY720 and this was coincident with the activation of caspase-3 (FIG. 8C). Intriguingly, Jurkat cells expressing S58A 14-3-3ζ-Myc consistently underwent commitment to apoptosis (as detected by the loss of TMRE staining) more slowly than those expressing wild type 14-3-3ζ-Myc (FIG. 8D). This suggests that the expression of S58A 14-3-3ζ slows the commitment to apoptosis in response to DMS, presumably by providing a pool of 14-3-3 resistant to phosphorylation.

Having observed that FTY720 behaved similarly to sphingosine and DMS in modulating 14-3-3 for phosphorylation in vitro we also examined the effect of S58A-14-3-3ζ expression on FTY720-induced apoptosis in Jurkat cells. Similarly to DMS, S58A 14-3-3ζ expressing cells showed a delayed mitochondrial permeability transition relative to wild type 14-3-3ζ expressing cells (FIG. 8D). These observations are important as it suggests that 14-3-3 is a down-stream target of both sphingosine and FTY720 and may determine the cell fate in response to both of these molecules.

### EXAMPLE 2

### Sphingolipid Modulation of 14-3-3 in Phosphorylation and Dimer Disruption

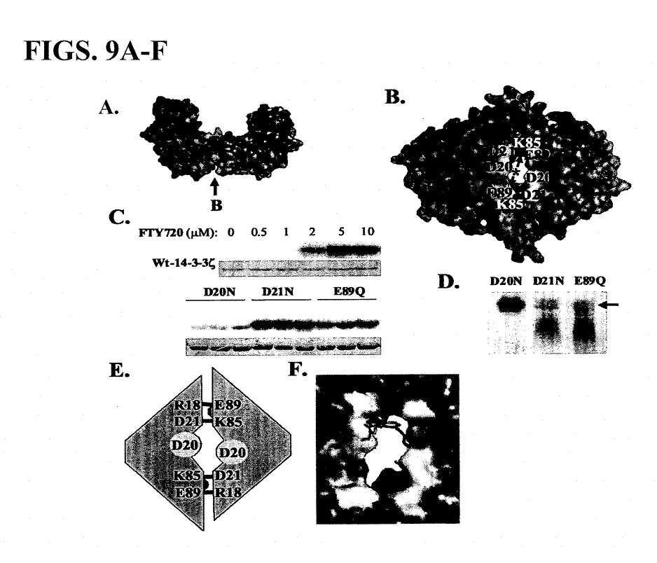

The dimer interface of 14-3-3 proteins lies at the bottom of the phospho-serine binding groove and comprises a central hole of around 10A in diameter in which the Ser58 phosphorylation site is positioned (FIG. 9B, indicated by *). The interaction of 14-3-3 protein with sphingolipids does not by itself bring about monomerisation of 14-3-3 (as determined by native-PAGE and gel filtration), but sphingolipid conformationally alters the protein to reveal the phosphorylation site. To determine how the sphingolipid controls phosphorylation site accessibility site-directed mutagenesis was performed to determine the residues of 14-3-3 important for lipid interaction. Since the basic nature of the sphingolipids is important for their effect, surface exposed acidic residues of 14-3-3 were subjected to conservative charge neutralisation substitutions. In particular, three substitutions, D20N, D21N and E89Q had dramatic effects on 14-3-3ζ phosphorylatability in response to lipid. These acidic residues are conserved across the 14-3-3 family and cluster at the base of the aperture on the under-side of the protein with respect to the phospho-serine peptide binding groove (FIG. 9B). In vitro phosphorylation studies with these recombinant mutant proteins show that, unlike the wild-type protein which shows increasing phosphorylatability by PKA catalytic subunit in response to FTY720 concentration, D2ON is only weakly phosphorylatable at any concentration of FTY720 whereas D21N and E89Q are strongly phosphorylatable even in the absence of FTY720 (FIG. 9C). The reduced phosphorylatability of the D2ON mutant protein suggests that the D20 residue is involved in interaction with FTY720 and its exposed position in the aperture fits with this possibility. Native-PAGE analysis of these mutants (FIG. 9D) revealed that D20N is dimeric whereas D21N and E89Q are not, explaining the relative accessibility of the Ser58 phosphorylation site in these two mutants. Thus these two conservative substitutions (D21N and E89Q) are individually sufficient to disrupt 14-3-3 dimer formation suggesting that through salt bridging they stabilise the dimer. The binding of basic FTY720 to D20 is sufficient to weaken the interaction of D21 and E89 with their salt bridging partners (K85 and R18 respectively) and thus expose the dimer interface and Ser58 for phosphorylation. In silico docking of FTY720 and sphingosine into the pocket encompassed by these residues (D20, D21 and E89) does indeed show a close fit of both molecules and suggests hydrogen-bonding between the polar heads of the lipids and both D20 and D21 (FIG. 9F & G), potentially breaking the salt-bridge between D21 and K85.

### EXAMPLE 3

### Analysing the Key Determinants of Sphingolipid Interaction in 14-3-3 Proteins

The non-phosphorylatable S58A mutant of 14-3-3(protects Jurkat cells from FTY720- and DMS-induced apoptosis (Woodcock et al. Cell. Signalling, 2010, 22, 1291-1299)^{(CIA6;CIB40)}. Mutants of D20, the residue identifieid in 14-3-3 as a sphingolipid binding residue should similarly provide protection against apoptosis. The 14-3-3ζ D20N mutant is expressed in Jurkat cells and use FTY720 and DMS as apoptotic stimuli and follow the rate of apoptosis in time course studies (as performed with the S58A mutant previously).

The D20 residue is conserved in all 14-3-3s and in their structures, as in l4-3-3ζ (FIG. 9B), bulges into the aperture separating the monomer subunits. To analyse the functioning of D20 in sphingolipid-induced modulation in different isoforms, charge reversal of this residue in other isoforms is carried out and sphingolipid-induced phosphorylation is assessed in vitro. Mutants are assessed for their dimeric status by native-PAGE (FIG. 9F) and cross-linking (Woodcock et al. 2003, J. Biol. Chem. 278:36323-36327) and gross perturbations in 14-3-3 structure are assessed by the ability to bind phospho-14-3-3 binding site peptide using a pull-down assay (Woodcock el al. 2003, J. Biol. Chem. 278:36323-36327). If other isoforms are rendered insensitive to sphingolipid through mutation these are expressed and analysed in the Jurkat apoptosis model (as above).

To gain further insight into the FTY720 binding region(s) in 14-3-3 proteins, the binding site on different 14-3-3 isoforms can be footprinted using photoreactive azido-FTY720 (Ubai et al. Anticancer Res., 2007, 27, 75-88). This molecule can be covalently cross-linked to proximal amino acid residues after UV irradiation and this approach has been previously used using azido-ATP to map the ATP binding site of SK1(Pitson et al. 2002, J. Biol. Chem. 277:49545-49553). Azido-FTY720 can be cross-linked to recombinant 14-3-3 protein isoforms in the presence or absence of excess non-photoreactive FTY720 to provide a control for specificity. This information allows analysis of residues in 14-3-3 in close proximity to bound azido-FTY720. These residues can also be targeted by site-directed mutagenesis and analysed for FTY720-induced phosphorylation (as above).

### EXAMPLE 4

### Analysing the Determining of 14-3-3 Protein Dimer Disruption

These first to show how exquisitely sensitive 14-3-3 dimers are to loss of a single salt bridge in the dimer interface (FIG. 9). Since the sphingolipids interfere with the salt bridges holding 14-3-3 dimers together, it is important to have means to analyse the functional salt bridge interactions involved in dimer stabilisation. As depicted in FIG. 9E, two salt bridges are important for dimer stability in 14-3-3: D21 with K85 and R18 with E89⁷. These residues are conserved across isoforms and with the exception of 14-3-3σ all isoforms readily form heterodimers (Yang et al. Proc. Nall. Acad. Sci. U.S.A., 2006, 103, 17237-17242; Chaudhri et al. Biochem. Biophys. Res. Comm., 2003, 300, 679-685). Site-directed mutagenesis has already revealed the importance of D21 and E89 for dimer formation in 14-3-3ζ (FIG. 9D). To further analyse the critical determinants of dimer formation mutation of the reciprocal residues of the salt bridges in 14-3-3C (R18Q and K85Q) can be performed. Additionally E5 and K74, which to, form a third salt bridge in the dimer interface, can be mutated although these residues are less well conserved in other isoforms and located further from the aperture containing the Ser58 phosphorylation site. Mutant proteins can be assessed for homo-dimerisation by native-PAGE, their ability to be phosphorylated in the presence or absence of sphingolipid. Paired charge reversal mutations can also be generated to study the pairing of the salt bridges ie. D21K can be combined with K85D and analysed for dimer formation.

To address how mutations that effect homo-dimerisation alter the ability of the mutant to hetero-dimerise (ie. where only one monomer unit is mutant) MEF cells from 14-3-3ζ knock-out mice can be used. These cells are readily transfectable and permissive for over-expression from pcDNA3-based expression constructs. The advantage of these cells is that transfected 14-3-3ζ can be expressed .in the absence of endogenous 14-3-3ζ and therefore after immunoprecipitation the pattern of hetero-dimer formation with endogenous isoforms can be analysed by immuno-blotting. To assess the stability of dimer-defective mutant 14-3-3 proteins, the expression of mutant 14-3-3 protein in the presence and absence of proteosomal inhibitors such as MG132 can be determined. For mutations that are significantly unstable (less than 20% expression of wild type 14-3-3) the protein half-life can be determined using cycloheximide treatment to block de novo protein synthesis.

### EXAMPLE 5

### Identifying Molecules that Target Dimeric 14-3-3 Disruption

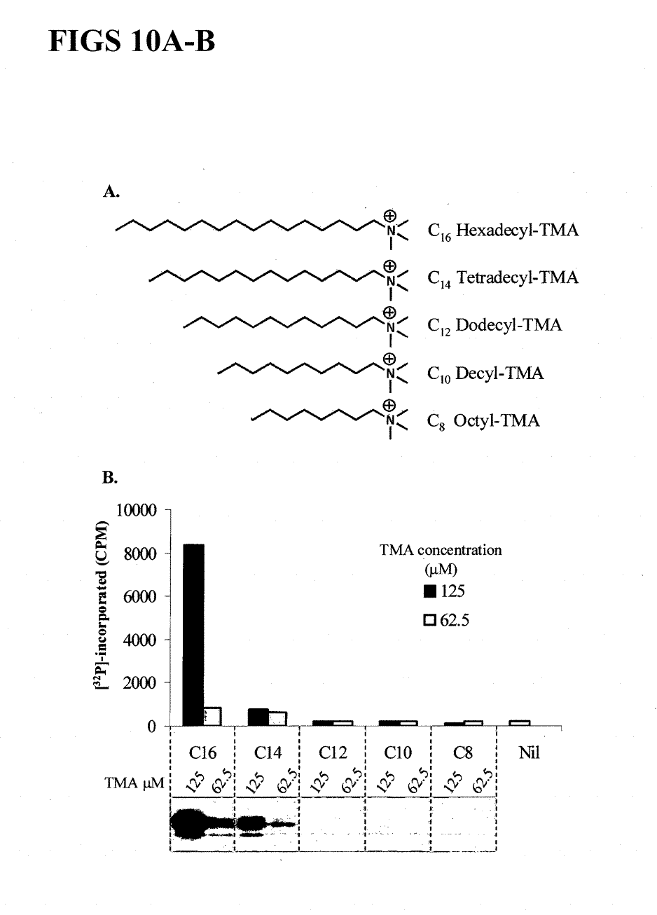

The assay to identify small molecules that modulate 14-3-3 for phosphorylation is based on the determination that sphingosine binds and modulates 14-3-3 for phosphorylation by kinases. As shown in FIG. 10, cationic hydrocarbons of varying length were tested for the ability to modulate 14-3-3ζ for phosphorylation by PKA, based on their chemical similarity to sphingosine. The molecules with longer acyl chains (C₁₆ and C₁₄) were capable of facilitating 14-3-3 phosphorylation.

A screen of lipids capable of conferring phosphorylatability on 14-3-3 was carried out. The focus was on lipids with similar structure to sphingosine.

### Structures of Sphingolipids and Analogues Tested for Ability to Modulate 14-3-3 for Phosphorylation

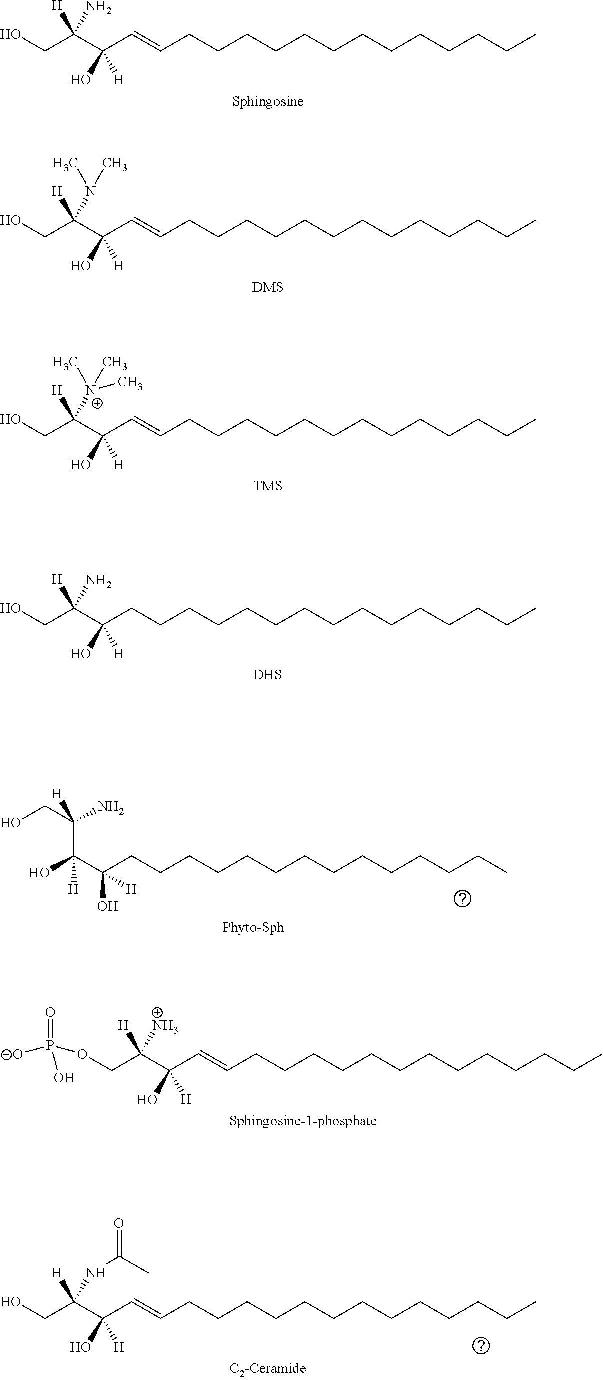

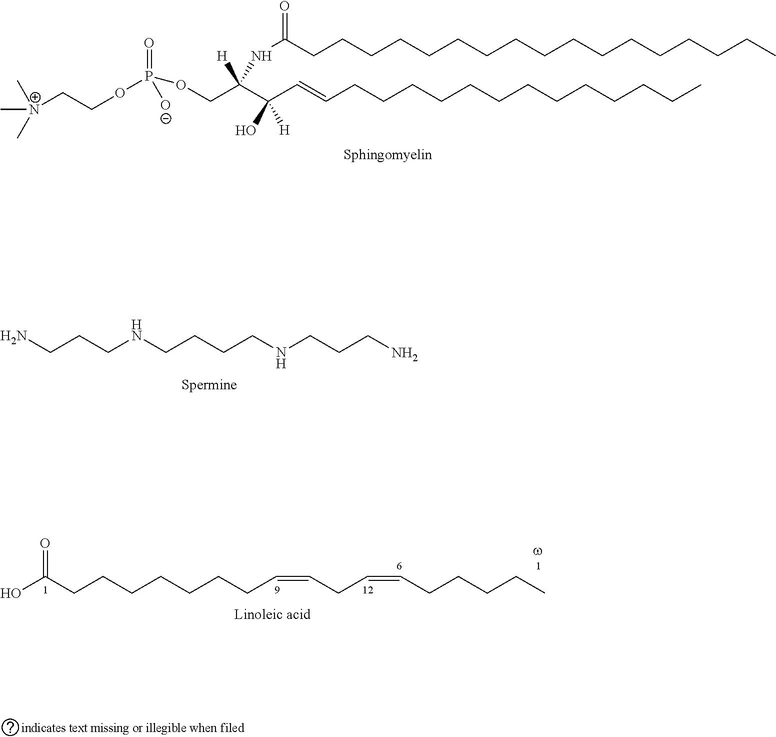 [SMILES: CCCCCCCCCCCCC/C=C/[C@@H](O)[C@H](COP(=O)([O-])OCC[N+](C)(C)C)NC(=O)CCCCCCCCCCCCCCCCC ; NCCCNCCCCNCCCN ; CCCCC/C=C\C/C=C\CCCCCCCC(=O)O]

Substrate modulation of 14-3-3 was found to be achieved using non-acylated (i.e. non conjugated to hydrocarbon chain at the primary. amine (basic sphingolipids that are not phosphorylated. The lipid modulation of 14-3-3 occurs with lipids carrying a net positive charge, indicating that basic sphingosines interact directly with the acidic 14-3-3 substrate.

Those skilled in the art will appreciate that the invention described herein is susceptible to variations and modifications other than those specifically described. It is to be understood that the invention includes all such variations and modifications. The invention also includes all of the steps, features, compositions and compounds referred to or indicated in this specification, individually or collectively, and any and all combinations of any two or more of said steps or features.

### BIBLIOGRAPHY

Bokoch et al. 1998, J. Biol. Chem. 273:8137-44

Chaudhri et al. Biochem. Biophys. Res. Comm., 2003, 300:679-685

Contreras et al. 2006, Biophys. J. 90:4085-4092

Cuvillier et al. 2000, J. Biol. Chem. 275:15691-15700

Cuvillier, O. 2002, Biochim. Biophys. Acta 1585:153-162

Davis et al. 1988, J. Biol. Chem. 263:5373-79

Denning et al. 2002, Cell Death & Differentiation 9:40-52

Fu et al. 2000, Ann. Rev. Pharma. Tox. 40:617-47

Gardino et al., 2006, Seminars in Cancer Biology 16:173-182

Gu et al. 2005, FEBS Letters 580:305-310

Hamaguchi et al. 2003, J. Biol. Chem. 278:41557-41565

Igarashi 1997, J. Biochem. 122:1080-87

Jones et al. 1995, J. Mol. Biol. 245:375-384

King et al. 2000, J. Biol. Chem. 275:18108-18113

Ma et al. 2005, J. Biol. Chem. 280:26011-26017

Mancini et al. J. Pharmacol. Exp. Ther., 2011, 336:596-604

Masters and Fu 2001, J. Biol. Chem. 276:45193-45200

McDonald et al. 1991, J. Biol. Chem. 266:21773-776

Megidish et al. 1998, J. Biol. Chem. 273:21834-21845

Nagahara et al. Br. J. Pharmcol. (2002) 137:953

Pham et al. 2008, Biotechniques, 45:155-162.

Pitson et al. 2002, J. Biol. Chem. 277:49545-49553

Powell et al. 2002, J. Biol. Chem. 277:21639-42.

Powell et al. 2003, MCB 23:5376-5387

Preisinger et al. 2004, J. Cell Biol. 164:1009-1020

Sitailo et al. 2004, J. invest. Dermatol. 123:434-443

Smith et al. 2000, Methods Enz. 312:361-373

Tzivion and Avruch 2002, J. Biol. Chem. 277:3061-64

Tzivion et al. 1998, Nature 394:88-92

Ubai et al. Anticancer Res., 2007, 27:75-88

Woodcock et al 2003, J. Biol. Chem. 278:36323-36327

Woodcock et al. Cell. Signalling, 2010, 22:1291-1299

Wu et al. Angew. Chem. Int. Ed, 2010, 49:6528-6532

Xing et al. 2000, EMBO 1 19:349-58

Yaffe 2002, FEBS Letts. 513:53-57

Yang et al. Proc. Natl. Acad. Sci. USA., 2006, 103:17237-17242

Zha et al. 1996, Cell 87:619-628

Zhang et a/. 1999, PNAS USA 96:8511-15

1-37. (canceled)

38. A method of modulating protein 14-3-3 or homologue or variant functionality in a mammal said method comprising administering to said mammal an effective amount of an agent for a time and under conditions sufficient to either modulate the interaction of sphingosine or homologue or variant with protein 14-3-3 or mimic the sphingosine interaction wherein agonising said interaction facilitates phosphorylation of Ser58 or analogous residue thereby inhibiting the functionality of said protein 14-3-3 and wherein antagonising said interaction inhibits phosphorylation of Ser58 or analogous residue thereby maintaining said protein 14-3-3 functionality.

39. A method for the treatment or prophylaxis of a condition in a mammal, which condition is characterised by inappropriate protein 14-3-3 or homologue or variant functionality, said method comprising administering to said mammal an effective amount of an agent which either modulates the interaction of sphingosine or homologue or variant with said protein 14-3-3 or which mimics the sphingosine interaction wherein agonising said interaction facilitates phosphorylation of Ser58 or analogous residue and inhibits the functionality of said protein 14-3-3 and wherein antagonising said interaction inhibits phosphorylation of Ser58 or analogous residue and maintains said protein 14-3-3 functionality.

40. The method according to claim 39, wherein said condition is characterised by inappropriate cellular division, cell growth, cell death or cellular apoptosis in a mammal, said method comprising administering to said mammal an effective amount of an agent which either modulates the interaction of sphingosine or homologue or variant with protein 14-3-3 or which mimics the sphingosine interaction, wherein agonising said interaction induces cellular apoptosis and wherein antagonising said interaction inhibits cellular apoptosis.

41. The method according to claim 38, wherein said serine 58 or analogous residue is selected from the list consisting of:
(i) Ser58 of protein 14-3ζ,
(ii) Ser59 of protein 14-3-3η;
(iii) Ser59 of protein 14-3-3γ;
(iv) Ser59 of protein 14-3-3ε; and
(v) Ser60 of protein 14-3-3β.

42. The method according to claim 38, wherein modulating the interaction between sphingosine and protein 14-3-3 or mimicking the sphingosine interaction is achieved by introducing into a mammal any one of:
(i) a molecule which mimics sphingosine or derivative, homologue, analogue or mimetic thereof;
(ii) the proteinaceous form of sphingosine or derivative, analogue, homologue or mimetic thereof;
(iii) a proteinaceous or non-proteinaceous molecule which antagonises the interaction between protein 14-3-3 and sphingosine;
(iv) a proteinaceous or non-proteinaceous molecule which agonises the interaction between protein 14-3-3 and sphingosine.

43. The method according to claim 42, wherein said sphingosine mimetic interacts with one or more of protein 14-3-3 residues D20, D21 , E89, K85, R18, E5, K74, R55 or analogous residue.

44. The method according to claim 43, wherein said protein 14-3-3 is selected from:
(i) protein 14-3-3γ and said residues are R19, D21, D22, R56, K88 or E92;
(ii) protein 14-3-3β and said residues are R20, D22, D23, R57, K87 or E91 ;
(iii) protein 14-3-3η and said residues are R19, D21, D22, R56, K88 or E92;
(iv) protein 14-3-3ε and said residues are R19, D21, E22, R56 or E92;
(v) protein 14-3-3τ and said residues are R18, D20, D21, R55, K85, or E89;
(vi) protein 14-3-3σ and said residues are R18, E20, D21, K87 or E91; and
(vii) protein 14-3-3ζ and said residues are D20, D21, E89, K85, R18, E5 or E91.

45. The method according to claim 42, wherein said molecule is non-phosphorylated and non-acylated.

46. The method according to claim 42, wherein said agonist is a sphingosine mimetic represented by formula (I): 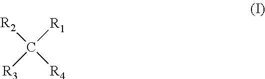
R₁ represents a saturated or unsaturated, branched or linear optionally substituted C₆-C₂₈ aliphatic group;
R₂ represents a C₂-C₄ alkyl substituted from 1 to 3 times with groups independently selected from —(CH₂)ₘOR′ where R′ is H or C₁₋₅ alkyl, —(CH₂)ₘCOOR″ where R″ is H or C₁₋₅ alkyl, —(CH₂)ₘNH₂, —(CH₂)ₘNH₃, —(CH₂)ₘN(C₁₋₆alkyl)₂, —(CH₂)ₘCHNH(COR′″)—(CH₂)ₚOH, —(CH₂)ₘN(C₁₋₆alkyl)₃, —(CH₂)ₘP(O)(OH)₂, —(CH)(—NHC(O)—C₁-C₂₈alkyl)-(CH₂)ₚ—O—P(O)(OH)—O—(CH₂)ₘ—NH₃, and —(CH)(—NHC(O)—C₁-C₂₈alkyl)-(CH₂)ₚ—O—P(O)(OH)—O—(CH₂)ₘ—N(C₁-C₄alkyl)₃;
R₃ and R₄ independently represent H or —(CH₂)ₙOR′″ where R′″ is H or C₁₋₃ alkyl;
m is 0, 1, or 2;
n is 0, 1, or 2;
p is 1 or 2;
or a pharmaceutically acceptable salt thereof.

47. The method according to claim 43, wherein said antagonist is:
(i) a molecule which competitively inhibits the sphingosine 14-3-3 interaction;
(ii) an antibody directed to protein 14-3-3 or sphingosine;
(iii) an miRNA, siRNA, antisense nucleic acid, such as antisense RNA directed to either the protein 14-3-3 or sphingosine nucleic acid molecules;
(iv) an aptamer directed to either protein 14-3-3 or sphingosine;
(v) a ribozyme, DNAzyme or molecule suitable for use in co-suppression.

48. The method according to claim 39, wherein said condition is a neoplastic condition or an inflammatory condition and said protein 14-3-3 activity is inhibited, a neurodegenerative condition characterized by unwanted apoptosis and said protein 14-3-3 activity is maintained, a cardiac disease characterized by unwanted tissue death and said protein 14-3-3 is maintained, and a condition in which angiogenesis is required to occur.

49. The method according to claim 48, wherein said neoplastic condition is a nervous system tumour, retinoblastoma, neuroblastoma and other paediatric tumour, head and neck cancer including squamous cell cancer, breast and prostate cancer, lung cancer including both small and non-small cell lung cancer, kidney cancer including renal cell adenocarcinoma), brain cancer, lung cancer, stomach cancer, oesophagogastric cancers, hepatocellular carcinoma, pancreaticobiliary neoplasia including adenocarcinoma and islet cell tumour, colorectal cancer, cervical and anal cancer, uterine and other reproductive tract cancer, urinary tract cancer including urinary tract cancer of the ureter and bladder, germ cell tumour including testicular germ cell tumour or ovarian germ cell tumour, ovarian cancer including ovarian epithelial cancer, carcinomas of unknown primary, human immunodeficiency associated malignancies including Kaposi's sarcoma, lymphoma, leukemia, malignant melanoma, sarcoma, endocrine tumour including endocrine tumours of the thyroid gland, mesothelioma and other pleural tumour, neuroendocrine tumour and carcinoid tumours; said inflammatory condition is rheumatoid arthritis, atherosclerosis, asthma, autoimmune disease or inflammatory bowel disease; said neurodegenerative condition is Parkinson's disease, Alzheimer's disease. Creutzfeldt-Jakob disease or transmissible spongiform encephalopathies, and said cardiac disease is myocardial infarction, angina, diabetic cardiomyopathy, hibernating myocardium in chronic ischaemia.

50. A method of modulating cellular apoptosis in a mammal said method comprising administering to said mammal an effective amount of an agent for a time and under conditions sufficient to either modulate the interaction of sphingosine or homologue or variant with protein 14-3-3 or mimic the interaction of sphingosine or homologue or variant with protein 14-3-3 wherein agonising said interaction induces apoptosis and wherein antagonising said interaction inhibits apoptosis.

51. A method for identifying an agent which facilitates the phosphorylation of protein 14-3-3 at Ser58 or analogous residue said method comprising:
(i) contacting a putative agent with protein 14-3-3 and a serine kinase or catalytic subunit thereof and screening for protein 14-3-3 phosphorylation at Ser58 or analogous residue; and
(ii) identifying the putative agent as agent which facilitates the phosphorylation of protein 14-3-3 at Ser58 or analogous residue on the basis of increased phosphorylation of the protein 14-3-3 at Ser58 or analogous residue.

52. The method according to claim 51, wherein said agent is a sphingosine mimetic.

53. The method according to claim 51, wherein said serine kinase is protein kinase A, PKCδ, AKT/PKB, MAPKAP-2, or SOK-1/YSK-1.

54. The method according to claim 51, wherein said screening for protein 14-3-3 phosphorylation comprises use of an assay selected from the list consisting of:
(i) SDS-PAGE analysis after [³²P]-γATP incorporation
(ii) P81 paper scintillation counting after radiolabel incorporation;
(iii) immunoblotting using a phospho-Ser58 antibody;
(iv) scintillation proximity assay after [³³P]-γATP incorporation; or
(v) ELISA using a phospho-Ser58 antibody.

55. The method according to claim 51, wherein the screening further comprises screening for conversion of dimeric protein 14-3-3 to monomeric protein 14-3-3 and/or screening for whether the putative agent mimics the interaction of sphingosine with the protein 14-3-3 and/or whether the putative agent agonises the interaction of sphingosine with the protein 14-3-3.

56. A method for identifying an agent which antagonises the phosphorylation of protein 14-3-3 at Ser58 or analogous residue said method comprising:
(i) contacting a putative agent with protein 14-3-3 and sphingosine;
(ii) contacting the putative agent, the protein 14-3-3 and the sphingosine of (i) with a serine kinase or catalytic subunit thereof, and screening for protein 14-3-3 phosphorylation at Ser58 or analogous residue; and
identifying the putative agent as an agent which antagonises the phosphorylation of protein 14-3-3 at Ser58 or analogous residue on the basis of a reduction in the level of phosphorylation of the protein 14-3-3 at the Ser58 or analogous residue.

57. A method for identifying an agent for treating a condition in a mammal characterised by unwanted or inappropriate cellular division or cell growth, said method comprising:
contacting a putative agent with protein 14-3-3 and a serine kinase or catalytic subunit thereof and screening for phosphorylation of the protein 14-3-3 at Ser58 or analogous residue; and
identifying the putative agent as an agent for treating a condition characterised by unwanted or inappropriate cellular division or cell growth by the ability of the putative agent to promote phosphorylation of the protein 14-3-3 at the Ser58 or analogous residue.

58. The method according to claim 57, wherein the screening further comprises screening for conversion of dimeric protein 14-3-3 to monomeric protein 14-3-3 and/or screening for whether the putative agent mimics the interaction of sphingosine with the protein 14-3-3 and/or whether the putative agent agonises the interaction of sphingosine with the protein 14-3-3.
